(19) United States
(12) Patent Application Publication
(10) Pub. No.: US 2005/0256182 A1
(43) Pub. Date: Nov. 17, 2005

(54) Formulations of anti-pain agents and methods of using the same

(76) Inventors: Diane E. Sutter, Cary, NC (US); Scott A. Kaestner, Durham, NC (US); Ronald J. Pettis, Cary, NC (US)

Correspondence Address: JONES DAY, 51 Louisiana Aveue, N.W, WASHINGTON, DC, 20001-2113, US

(21) Appl. No.: 11/126,335

(22) Filed: May 11, 2005

Related U.S. Application Data
(60) Provisional application No. 60/570,064, filed on May 11, 2004.
(60) Provisional application No. 60/592,101, filed on Jul. 29, 2004.

(51) Int. Cl. A61K 31/405

(52) U.S. Cl. 514/419

(57) ABSTRACT

The present invention relates to novel anti-pain formulations and methods of their delivery. Anti-pain agents delivered in accordance with the methods of the invention have an improved clinical utility and therapeutic efficacy relative to other drug delivery methods, including oral, intramuscular and subcutaneous delivery. The methods of the present invention provide benefits and improvements over conventional drug delivery methods including dose sparing, increased drug efficacy, reduced side effects.

This application claims priority to U.S. Provisional Application Nos. 60/570,064, filed May 11, 2004 and 60/592,101, filed Jul. 29, 2004, both of which are incorporated herein in their entireties by reference.

### 1. FIELD OF THE INVENTION

The present invention relates to methods and devices for dermal delivery of therapeutically and/or prophylactically effective amounts of agents for management of pain, particularly anti-migraine agents, more particularly sumatriptan succinate. In accordance with the present invention, the anti-migraine agents are deposited into the intradermal compartment and/or junctional space, i.e., between intradermal and subcutaneous compartments, of a subject's skin. Agents delivered in accordance with the methods of the invention have an improved clinical utility and therapeutic efficacy relative to other drug delivery methods, including intraperitoneal, intramuscular and subcutaneous delivery. The methods of the present invention provide benefits and improvements over conventional drug delivery methods including dose sparing, increased drug efficacy, reduced side effects.

### 2. BACKGROUND OF THE INVENTION

The following description includes information that may be useful in understanding the present invention. It is not an admission that any of the information provided herein is prior art to the presently claimed inventions, or relevant, nor that any of the publications specifically or implicitly referenced are prior art.

2.1 Pain

Pain is the leading symptom of many different disorders and is defined as an unpleasant sensory and emotional experience associated with actual or potential tissue damage or described in terms of such damage. Classification of Chronic Pain, International Association for the Study of Pain (IASP) Task Force on Taxonomy, Merskey H, Bogduk N, eds., IASP Press: Seattle, 209-214, 1994. Because the perception of pain is highly subjective, it is one of the most difficult pathologies to diagnose and treat effectively. Pain leads to severe impairment of functional ability, which compromises the working, social, and family lives of sufferers. Around five percent of the adult population is estimated to suffer from pain sufficiently severe to cause significant disability. Chojnowska E, Stannard C. Epidemiology of Chronic Pain, Chapter 2, pp 15-26: T. S. Jensen, P. R. Wilson, A. S. C. Rice eds., Clinical Pain Management Chronic Pain, Arnold, London, 2003.

In most pain conditions, there is increased neural input from the periphery. Sensory nerve impulses travel via the axons of primary afferent neurons to the dorsal horn of the spinal cord, where they propagate nerve impulses to dorsal horn neurons by releasing excitatory amino acids and neuropeptides at synapses. Dorsal horn projection neurons process and transfer the information about a peripheral stimuli to the brain via ascending spinal pathways. Mannion, R. J. and Woolf, C. J., Clin. J of Pain 16:S144-S156 (2000).

The firing of dorsal horn projection neurons is determined not only by the excitatory input they receive, but also by inhibitory input from the spinal cord and higher nerve centers. Several brain regions contribute to descending inhibitory pathways. Nerve fibers from these pathways release inhibitory substances such as endogenous opioids, γ-aminobutyric acid (GABA), and serotonin at synapses with other neurons in the dorsal horn or primary afferent neurons and inhibit nociceptive transmission. Peripheral nerve injury can produce changes in dorsal horn excitability by down-regulating the amount of inhibitory control over dorsal horn neurons through various mechanisms.

Repeated or prolonged stimulation of dorsal horn neurons due to C-nociceptor activation or damaged nerves can cause a prolonged increase in dorsal horn neuron excitability and responsiveness that can last hours longer than the stimulus. Sensitization of the dorsal horn neurons increases their excitability such that they respond to normal input in an exaggerated and extended way. It is now known that such sustained activity in primary afferent C-fibers leads to both morphological and biochemical changes in the dorsal horn which may be difficult to reverse. Several changes in the dorsal horn have been noted to occur with central sensitization: (i) an expansion of the dorsal horn receptive field size so that a spinal neuron will respond to noxious stimuli outside the region normally served by that neuron; (ii) an increase in the magnitude and duration of the response to a given noxious stimulus (hyperalgesia); (iii) a painful response to a normally innocuous stimulus, for example, from a mechanoreceptive primary afferent Aβ-fibre (allodynia); and (iv) the spread of pain to uninjured tissue (referred pain). Koltzenburg, M. Clin. J. of Pain 16:S131-S138 (2000); Mannion, R. J. and Woolf, C. J., Clin. J of Pain 16:S144-S156 (2000).

Central sensitization may explain, in part, the continuing pain and hyperalgesia that occurs following an injury and may serve an adaptive purpose by encouraging protection of the injury, during the healing phase. Central sensitization however can persist long after the injury has healed thereby supporting chronic pain. Sensitization also plays a key role in chronic pain, helping to explain why it often exceeds the provoking stimulus, both spatially and temporally, and may help explain why established pain is more difficult to suppress than acute pain. Koltzenburg, M. Clin. J. of Pain 16:S131-S138 (2000).

Accordingly, safe and effective methods for the treatment, prevention, modification or management of pain are needed.

2.1.1 Types of Pain

2.1.1.1 Nociceptive Pain

Nociceptive pain is elicited when noxious stimuli such as inflammatory chemical mediators are released following tissue injury, disease, or inflammation and are detected by normally functioning sensory receptors (nociceptors) at the site of injury. Koltzenburg, M. Clin. J of Pain 16:S131-S138 (2000). Clinical examples of nociceptive pain include, but are not limited to, pain associated with chemical or thermal burns, cuts and contusions of the skin, osteoarthritis, rheumatoid arthritis, tendonitis, and myofascial pain. Nociceptors (sensory receptors) are distributed throughout the periphery of tissue. They are sensitive to noxious stimuli (e.g., thermal, mechanical, or chemical) which would damage tissue if prolonged. Activation of peripheral nociceptors by such stimuli excites discharges in two distinct types of primary afferent neurons: slowly conducting unmyelinated c-fibers and more rapidly conducting, thinly myelinated Aδ fibers. C-fibers are associated with burning pain and Aδ fibers with stabbing pain. Koltzenburg, M. Clin. J of Pain 16:S131-S138 (2000); Besson, J. M. Lancet 353:1610-15 (1999); Johnson, B. W. Pain Mechanisms: Anatomy, Physiology and Neurochemistry, Chapter 11 in Practical Management of Pain ed. P. Prithvi Raj. (3^{rd} Ed., Mosby, Inc. St Louis, 2000). Most nociceptive pain involves signaling from both Aδ and c-types of primary afferent nerve fibers.

Peripheral nociceptors are sensitized by inflammatory mediators such as prostaglandin, substance P, bradykinin, histamine, and serotonin, as well as by intense, repeated, or prolonged noxious stimulation. In addition, cytokines and growth factors (e.g., nerve growth factor) can influence neuronal phenotype and function. Besson, J. M. Lancet 353:1610-15 (1999).

When sensitized, nociceptors exhibit a lower activation threshold and an increased rate of firing, which means that they generate nerve impulses more readily and more frequently. Peripheral sensitization of nociceptors prays an important role in spinal cord dorsal horn central sensitization and clinical pain states such as hyperalgesia and allodynia.

Inflammation also appears to have another important effect on peripheral nociceptors. Some C-nociceptors do not normally respond to any level of mechanical or thermal stimuli, and are only activated in the presence of inflammation or in response to tissue injury. Such nociceptors are called “silent” nociceptors, and have been identified in visceral and cutaneous tissue. Besson, J. M. Lancet 353:1610-15 (1999); Koltzenburg, M. Clin. J. of Pain 16:S131-S138 (2000).

Differences in how noxious stimuli are processed across different tissues contribute to the varying characteristics of nociceptive pain. For example, cutaneous pain is often described as a well-localized sharp, prickling, or burning sensation whereas deep somatic pain may be described as diffuse, dull, or an aching sensation. In general, there is a variable association between pain perception and stimulus intensity, as the central nervous system and general experience influence the perception of pain.

2.1.1.2 Neuropathic Pain

Neuropathic pain reflects injury or impairment of the nervous system, and has been defined by the IASP as “pain initiated or caused by a primary lesion or dysfunction in the nervous system”. Classification of Chronic Pain, International Association for the Study of Pain (IASP) Task Force on Taxonomy, Merskey H, Bogduk N, eds., IASP Press: Seattle, 209-214, 1994. Some neuropathic pain is caused by injury or dysfunction of the peripheral nervous system. As a result of injury, changes in the expression of key transducer molecules, transmitters, and ion channels occur, leading to altered excitability of peripheral neurons. Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000). Clinical examples of neuropathic pain include, but are not limited to, pain associated with diabetic neuropathy, postherpetic neuralgia, trigeminal neuralgia, and post-stroke pain.

Neuropathic pain is commonly associated with several distinct characteristics, such as pain which may be continuous or episodic and is described in many ways, such as burning, tingling, prickling, shooting, electric-shock-like, jabbing, squeezing, deep aching, or spasmodic. Paradoxically partial or complete sensory deficit is often present in patients with neuropathic pain who experience diminished perception of thermal and mechanical stimuli. Abnormal or unfamiliar unpleasant sensations (dysaesthesias) may also be present and contribute to suffering. Other features are the ability of otherwise non-noxious stimuli to produce pain (allodynia) or the disproportionate perception of pain in response to supra-threshold stimuli (hyperalgesia). Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000); Attal, N. Clin. J. of Pain 16:S118-S130 (2000).

2.1.2 Visceral Pain

Visceral pain has been conventionally viewed as a variant of somatic pain, but may differ in neurological mechanisms. Visceral pain is also thought to involve silent nociceptors, visceral afferent fibers that only become activated in the presence of inflammation. Cervero, F. and Laird J. M. A. , Lancet 353:2145-48 (1999).

Certain clinical characteristics are peculiar to visceral pain: (i) it is not evoked from all viscera and not always linked to visceral injury; (ii) it is often diffuse and poorly localized, due to the organization of visceral nociceptive pathways in the central nervous system (CNS), particularly the absence of a separate visceral sensory pathway and the low proportion of visceral afferent nerve fibers; (iii) it is sometimes referred to other non-visceral structures; and (iv) it is associated with motor and autonomic reflexes, such as nausea. Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000); Cervero, F. and Laird J. M. A., Lancet 353:2145-48 (1999).

Headaches can be classified as primary and secondary headache disorders. The pathophysiology of the two most common primary disorders, migraine and tension-type headache, is complex and not fully understood. Recent studies indicate that nociceptive input to the CNS may be increased due to the activation and sensitization of peripheral nociceptors, and the barrage of nociceptive impulses results in the activation and sensitization of second- and third-order neurons in the CNS. Thus, it is likely that central sensitization plays a role in the initiation and maintenance of migraine and tension-type headache. Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000). Migraine headaches are known to produce the most intense headaches reported. The pathophysiology of migraine headaches involve vasoconstriction and vasodilation. A variety of stress stimuli, including intense light, noise, anxiety, exertion, extremes of temperature, hormones, exhaustion, infection and trauma result in constriction of extracranial blood vessels. The vasoconstriction is followed by reflexive or sequential vasodilation, which subsequently spreads to intracranial vessels. It is during this latter phase that the patient feels the intense, throbbing headache characteristic of migraines. Increased levels of norepinephrine, serotonin, histamine, and the neuropeptides bradykinin and substance P, in addition to products of tissue anoxia, are considered to be the main endogenous pain producing molecules, accompanied by direct sensory nerve stimulation because of the stretching that accompanies vasoconstriction and dilation. Sumatriptan, (Imitrex, GlaxoSmithKline) currently on the market to treat migraine headaches is offered in three formulations: oral, nasal and injectable. Those who need immediate relief from the excruciating pain of a migraine headache prefer the injectable. The injectable is a 12 mg/ml solution with a therapeutic dose of 6 mg or 0.5 ml self-administered SC as a bolus injection. Each 0.5 ml contains 6 mg of the sumatriptan (base) as the succinate salt and 3.5 mg of sodium chloride in water for injection. The pH range of the solution is approximately 4.2 to 5.3 with an osmolality of 291 mOsmol. The current formulation is known to cause injection site reactions that include pain, redness, stinging in duration, contusion and swelling. There thus remains a need for more effective methods of treatment, prevention and management of migraine and associated conditions.

Post-operative pain, such as that resulting from trauma to tissue caused during surgery, produces a barrage of nociceptive input. Following surgery, there is an inflammatory response at the site of injury involving cytokines, neuropeptides and other inflammatory mediators. These chemical are responsible for the sensitization and increased responsiveness to external stimuli, resulting in, for example, lowering of the threshold and an increased response to supra-threshold stimuli. Together, these processes result in peripheral and central sensitization. Johnson, B. W., Pain Mechanisms: Anatomy, Physiology and Neurochemistry, Chapter 11 in Practical Management of Pain ed. P. Prithvi (Raj. 3^{rd} Ed., Mosby, Inc., St Louis, 2000).

Mixed pain is chronic pain that has nociceptive and neuropathic components. For example, a particular pain can be initiated through one pain pathway and sustained through a different pain pathway. Examples of mixed pain states include, but are not limited to, cancer pain and low back pain.

2.2 Drug Delivery

The importance of efficiently and safely administering pharmaceutical substances such as diagnostic agents and drugs has long been recognized. Although an important consideration for all pharmaceutical substances, obtaining adequate bioavailability of large molecules such as proteins that have arisen out of the biotechnology industry has recently highlighted this need to obtain efficient and reproducible absorption (Cleland et al., 2001 Curr. Opin. Biotechnol. 12: 212-219). The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e., delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal and/or junctional space. Furthermore, even though the dermis, in particular, the papillary dermis has been known to have a high degree of vascularity, prior to the instant invention it was not appreciated that one could take advantage of this high degree of vascularity to obtain an improved absorption profile for administered substances compared to subcutaneous administration.

Small drug molecules have been traditionally administered subcutaneously because they are rapidly absorbed after administration into the subcutaneous tissue and subcutaneous administration provides an easy and predictable route of delivery. However, the need for improving the pharmacokinetics of administration of small molecules has not been appreciated. Large molecules such as proteins are typically not well absorbed through the capillary epithelium regardless of the degree of vascularity of the targeted tissue. Effective subcutaneous administration for these substances has thus been limited.

One approach to administration beneath the surface to the skin and into the region of the intradermal and/or junctional space has been routinely used in the Mantoux tuberculin test. In this procedure, a purified protein derivative is injected at a shallow angle to the skin surface using a 27 or 30 gauge needle (Flynn et al., 1994 Chest 106:1463-5). A degree of uncertainty in placement of the injection can, however, result in some false negative test results. Moreover, the test has involved a localized injection to elicit a response at the site of injection and the Mantoux approach has not led to the use of intradermal and/or junctional injection for systemic administration of substances.

Some groups have reported on systemic administration by what has been characterized as “intradermal” injection. In one such report, a comparative study of subcutaneous and what was described as “intradermal” injection was performed (Autret et al., 1991 Therapie 46:5-8). The pharmaceutical substance tested was calcitonin, a protein of a molecular weight of about 3600. Although it was stated that the drug was injected intradermally, the injections used a 4 mm needle pushed up to the base at an angle of 60. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al., 1993 J. Pharm. Sci. 82.1175-1178). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as an intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus, there remains a continuing need for efficient and safe methods and devices for administration of pharmaceutical substances.

### 3. SUMMARY OF THE INVENTION

The present invention relates to methods and devices for intradermal and/or junctional delivery of therapeutically and/or prophylactically effective amounts of agents for management of pain, particularly anti-migraine agents, by depositing the agent into the intradermal and/or junctional compartment of a subject's skin. Preferred anti-migraine agents are triptan compounds. As used herein, “triptan compounds” refer to the group of chemical compounds that contain 2-(1H-indol-3-yl)-N,N-dimethylethanamine moiety. In accordance with this invention, the triptan compounds include, but are not limited to, almotriptan, zolmitriptan, rizatriptan, sumatriptan, naratriptan, or pharmaceutically acceptable salts thereof. Preferred salts are almotriptan malate, rizatriptan benzoate, sumatriptan succinate, and naratriptan hydrochloride. Most preferred compound is sumatriptan succinate. Although methods and formulations of the invention are described in connection with sumatriptan succinate by way of an example, the use of other anti-pain agents, in particular, other triptan compounds, are also encompassed and can be optimized based on the description using well-known methods in the art.

Agents delivered in accordance with the methods of the invention have an improved clinical utility and therapeutic efficacy relative to other drug delivery methods, including intraperitoneal, intramuscular and subcutaneous delivery.

The present invention relates to improved treatment, prevention, control and management of varying types and severities of pain and related syndromes, including but not limited to nociceptive pain, neuropathic pain, acute pain, chronic pain, nociceptive pain resulting from physical trauma (e.g., a cut or contusion of the skin; or a chemical or thermal burn), osteoarthritis, rheumatoid arthritis or tendonitis, myofascial pain, modifying mixed pain (i.e., pain with both nociceptive and neuropathic components), visceral pain; headache pain (e.g., migraine headache pain); mixed pain (i.e., chronic pain having nociceptive and neuropathic components); reflex neurovascular dystrophy; reflex dystrophy; sympathetically maintained pain syndrome; causalgia; Sudeck atrophy of bone; algoneurodystrophy; shoulder hand syndrome; post-traumatic dystrophy; autonomic dysfunction; cancer-related pain; phantom limb pain; fibromyalgia; myofascial pain; chronic fatigue syndrome; post-operative pain; spinal cord injury pain; central post-stroke pain; radiculopathy; sensitivity to temperature, light touch or color change to the skin (allodynia); pain from hyperthermic or hypothermic conditions; and other painful conditions (e.g., diabetic neuropathy, luetic neuropathy, postherpetic neuralgia, trigeminal neuralgia, or painful neuropathy induced iatrogenically by drugs such as vincristine, velcade or thalidomide).

In most preferred embodiments, the invention relates to the treatment, prevention and management of migraine and associated conditions, including but not limited to migraine without aura (“common migraine”), migraine with aura (“classic migraine”), migraine with typical aura, migraine with prolonged aura, familial hemiplegic migraine, basilar migraine, migraine aura without headache, migraine with acute-onset aura, opthalmoplegic migraine, retinal migraine, cluster headaches, chronic paroxysmal hemicrania, headache associated with vascular disorders, tension headache and pediatric migraine by intradermal and/or junctional delivery of agents for management of pain, particularly anti-migraine agents, more particularly sumatriptan succinate, to a subject, preferably humans, by directly targeting the dermal or junctional space whereby such method alters the pharmacokinetics (PK) and pharmacodynamics (PD) parameters of the administered agent. Thus, the methods of the invention are particularly useful for the treatment, prevention and/or management of migraine and associated conditions.

The present invention is based, in part, on the inventors' unexpected discovery that delivering sumatriptan succinate at higher concentrations than traditionally used and at lesser volumes to the intradermal (ID) and/or junctional compartment resulted in reduction in skin irritation (e.g., erythema, edema at the site of injection) compared to subcutaneous (SC) delivery while altering the PK and PD effects of the administered drug. Based on the reduction in skin irritation, delivery of sumatriptan succinate to the Intradermal and/or junctional space is expected to result in reduction of pain, and as a result, greater compliance as compared to conventional delivery to the subcutaneous or intramuscular compartment.

The present invention is also based, in part, on the inventors' unexpected discovery that delivering a novel formulation of sumatriptan succinate resulted in several benefits including, but are not limited to, reduction in mechanical pain and skin irritation, and minimization of spillover of the solution.

As used herein, intradermal administration is intended to encompass administration of agents for management of pain, particularly anti-migraine agents, more particularly sumatriptan succinate to the dermis in such a manner that the agent readily reaches the dermal vasculature, including both the circulatory and lymphatic vasculature, and is rapidly absorbed into the blood capillaries and/or lymphatic vessels to become systemically bioavailable. As used herein, junctional administration is intended to encompass administration of agents for management of pain, particularly anti-migraine agents, more particularly sumatriptan succinate to the junctional space between intradermal and subcutaneous compartments. Preferably, deposition of agents for management of pain, particularly anti-migraine agents, more particularly sumatriptan succinate predominately at a depth of at least about 0.3 mm, more preferably, at least about 0.4 mm and most preferably at least about 0.5 mm up to a depth of no more than about 3 mm, more preferably, no more than about 2.5 mm and most preferably no more than about 1.5 mm will result in rapid absorption of the agent. Preferably, agents for management of pain, particularly anti-migraine agents, more particularly sumatriptan succinate are delivered in accordance with the present invention at a depth of 1.5 mm, 2 mm or 3 mm.

Directly targeting the dermal or junctional space as taught by the invention provides more rapid onset of effects of agents for management of pain, particularly anti-migraine agents, more particularly sumatriptan succinate. Preferably, the formulations of agents for management of pain, particularly anti-migraine agents, more particularly sumatriptan succinate, are rapidly absorbed and systemically distributed via controlled Intradermal and/or junctional administration that selectively accesses the circulatory and lymphatic microcapillaries, thus the agent may exert their beneficial effects more rapidly than SC administration.

Delivering agents for management of pain, particularly anti-migraine agents, more particularly sumatriptan succinate to the intradermal and/or junctional compartment results in improved pharmacokinetics relative to conventional methods of such agent (e.g., sumatriptan succinate) delivery. According to the present invention, the term “improved pharmacokinetics” means increased bioavailability, decreased lag time (T_{lag}), decreased Tₘₐₓ, more rapid absorption rates, more rapid onset and/or increased Cₘₐₓ for a given amount of compound administered, compared to conventional delivery routes for agents for management of pain. Conventional delivery routes include delivery to SC or IM compartment, or oral delivery. In a preferred embodiment, “improved pharmacokinetics” means an enhancement in at least two of the following parameters: increased bioavailability, decreased lag time (T_{lag}), decreased Tₘₐₓ, more rapid absorption rates, more rapid onset and increased Cₘₐₓ.

As used herein, the term “bioavailability” means the total amount of a given dosage of the delivered substance that reaches the blood compartment. This is generally measured as the area under the curve in a plot of concentration vs. time. By “lag time” is meant the delay between the administration of the delivered substance and time to measurable or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a compound. However, numerical values can be determined more precisely by kinetic analysis using mathematical models and/or other means known to those of skill in the art.

By “enhanced absorption profile,” it is meant that absorption is improved over or greater than that obtained from conventional routes of delivery, as measured by such pharmacokinetic parameters. Conventional delivery routes include delivery to SC or IM compartment, or oral delivery. The measurement of pharmacokinetic parameters and determination of minimally effective concentrations are routinely performed in the art. Values obtained are deemed to be enhanced by comparison with a standard route of administration such as, for example, subcutaneous, intramuscular, or oral administration. In such comparisons, it is preferable, although not necessarily essential, that administration into the intradermal and/or junctional layer and administration into the reference site such as subcutaneous administration involve the same dose levels, i.e., the same amount and concentration of the agent as well as the same carrier vehicle and the same rate of administration in terms of amount and volume per unit time. Thus, for example, administration of a given agent into the dermis at a concentration such as 100 μg/mL and rate of 100 μL per minute over a period of 5 minutes would, preferably, be compared to administration of the same agent into the subcutaneous space at the same concentration of 100 μg/mL and rate of 100 μL per minute over a period of 5 minutes.

In accordance with the invention, administration to the Intradermal and/or junctional spaces of the skin can be achieved using, for example, microneedle-based injection and infusion systems or any other means known to one skilled in the art to accurately target the desired space. In accordance of the present invention, the terms “administration,” “delivery,” “depositing,” “targeting,” and “directly targeting,” when used in connection with the delivery of agents into a tissue compartment, are used interchangeably.

As used herein, and unless otherwise specified, the term “intradermal (ID) space” means the skin compartment known as the dermis, which is located beneath the epidermis. The dermis includes the papillary dermis and the reticular dermis. Typically, intradermal administration involves depositing an agent into the skin at a depth of from about 0.5 mm to about 2 mm, preferably from about 1 mm to about 2 mm.

As used herein, and unless otherwise specified, the term “junctional space” means the interface skin compartment that separates the reticular dermis and subcutaneous tissue. Typically, junctional administration of an agent involves depositing the agent into the skin at a depth of from about 2 mm to about 3 mm, preferably from about 2.5 mm to about 3 mm.

In accordance with the invention, the terms “space,” “compartment,” and “layer” are used interchangeably.

Using the methods of the invention, the pharmacokinetics of agents for management of pain, particularly anti-migraine agents, more particularly sumatriptan succinate, can be altered when compared to traditional methods of agents for management of pain, particularly anti-migraine agents, more particularly sumatriptan succinate delivery. Improved pharmacokinetic parameters using methods of the invention can be achieved using not only microdevice-based injection systems, but other delivery systems such as needle-less or needle-free ballistic injection of fluids or powders into the Intradermal and/or junctional space, Mantoux-type ID injection, enhanced ionotophoresis through microdevices, and direct deposition of fluid, solids, or other dosing forms into the skin.

Another benefit of the invention is to achieve more rapid systemic distribution and offset of agents for management of pain, particularly anti-migraine agents, more particularly sumatriptan succinate. The methods of the invention also help achieve higher bioavailabilities of agents for management of pain, particularly anti-migraine agents, more particularly sumatriptan succinate. The direct benefit is that ID and/or junctional administration with enhanced bioavailability allows equivalent biological effects while using less active agent. This results in direct economic benefit to the drug manufacturer and perhaps consumer. Likewise, higher bioavailability may allow reduced overall dosing and decrease the patient's side effects associated with higher dosing.

Yet another benefit of the invention is the attainment of higher maximum concentrations of agents for management of pain, particularly anti-migraine agents, more particularly sumatriptan succinate in the plasma. The inventors have found that agents for management of pain, particularly anti-migraine agents, more particularly sumatriptan succinate, administered in accordance with the methods of the invention is absorbed more rapidly, resulting in higher initial concentrations in the plasma. The more rapid onset allows higher C_{Max} values to be reached with lesser amounts of the agent.

Another benefit of the invention is removal of the physical or kinetic barriers invoked when agents for management of pain, particularly anti-migraine agents, more particularly sumatriptan succinate, is transdermally delivered. Direct Intradermal and/or junctional administration by mechanical means in contrast to transdermal delivery methods overcomes the kinetic barrier properties of skin, and is not limited by the pharmaceutical or physicochemical properties of agents for management of pain, particularly anti-migraine agents, more particularly sumatriptan succinate, or its formulation excipients.

These and other benefits of the invention are achieved by directly targeting the dermal vasculature and by controlled delivery of agents for management of pain, particularly anti-migraine agents, more particularly sumatriptan succinate, to the dermal or junctional space of skin. The inventors have found that by specifically targeting the intradermal and/or junctional space and controlling the rate and pattern of delivery, the pharmacokinetics exhibited by agents for management of pain, particularly anti-migraine agents, more particularly sumatriptan succinate, can be unexpectedly improved, and can in many situations be varied with resulting clinical advantage. Such pharmacokinetic control cannot be as readily obtained or controlled by other parenteral administration routes, except by IV access.

Using the methods of the present invention, agents for management of pain, particularly anti-migraine agents, more particularly sumatriptan succinate, may be administered as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

This invention also encompasses formulations comprising agents for management of pain, particularly anti-migraine agents, more particularly triptan compounds, and methods of administration of the formulations. Preferred anti-migraine agents are triptan compounds. As used herein, “triptan compounds” refer to the group of chemical compounds that contain 2-(1H-indol-3-yl)-N,N-dimethylethanamine moiety. In accordance with this invention, the triptan compounds include, but are not limited to, almotriptan, zolmitriptan, rizatriptan, sumatriptan, naratriptan, or pharmaceutically acceptable salts thereof. Preferred salts are almotriptan malate, rizatriptan benzoate, sumatriptan succinate, and naratriptan hydrochloride. Most preferred compound is sumatriptan succinate. Although formulations and methods of the invention are described in connection with sumatriptan succinate by way of an example, the use of other triptan compounds are also encompassed and can be optimized based on the description using well-known methods in the art.

The agents for management of pain, particularly anti-migraine agents, more particularly sumatriptan succinate, may be in any form suitable for intradermal and/or junctional delivery. In one embodiment, the agent of the invention is in the form of a flowable, injectable medium, i.e., a low viscosity formulation that may be injected in a syringe. The flowable injectable medium may be a liquid. Alternatively, the flowable injectable medium is a liquid in which particulate material is suspended, such that the medium retains its fluidity to be injectable and syringable, e.g., can be administered in a syringe. In most preferred embodiments, the invention encompasses a formulation of sumatriptan succinate, which meets volumetric limitations for intradermal (ID) or junctional injection, has a concentration of sufficient strength to provide the recommended dosage of sumatriptan (6 mg), and is physiologically acceptable for Intradermal and/or junctional administration (e.g., causes minimal dermal irritation at the injection site).

The agents for management of pain, particularly anti-migraine agents, more particularly sumatriptan succinate, of the present invention can be prepared as unit dosage forms. A unit dosage per vial may contain 0.1 to 0.5 mL of the formulation. In some embodiments, a unit dosage form of the formulations of the invention may contain 50 μL to 100 μL, 50 μL to 200 μL, or 50 μL to 500 μL of the formulation. If necessary, these preparations can be adjusted to a desired concentration by adding a sterile diluent to each vial.

In yet other preferred embodiments, the invention provides a formulation of sumatriptan succinate for intradermal and/or junctional delivery to enhance user acceptance of parenteral therapy by reducing the physiological and perception factors associated with injection and also provides pharmacological benefits including but not limited to reduced time to onset of systemic bioavailability and pharmacological action, e.g., reduced time to pain relief onset.

The invention provides an improved formulation of injectable sumatriptan succinate to make it acceptable for delivery to the intradermal and/or junctional space. The improved formulation may also be delivered via conventional routes of delivery including, but not limited to, delivery to SC and IM, or oral delivery. Formulation of the invention contains sumatriptan succinate at a higher concentration than conventionally used formulations, including, but limited to, greater than about 20 mg/ml, about 24 mg/ml, or about 30 mg/ml. In some embodiments, formulation of the invention contains sumatriptan succinate at a concentration of from about 20 mg/ml to about 60 mg/ml, from about 20 mg/ml to about 40 mg/ml, from about 25 mg/ml to about 40 mg/ml, from about 20 mg/ml to about 30 mg/ml, from about 23 mg/ml to about 35 mg/ml, or from about 25 mg/ml to about 30 mg/ml. The term “about,” as used herein, is used to denote that the concentrations are approximate. Specifically, the term “about” encompasses deviations of less than 2 mg, 1.5 mg, 1 mg, 0.5 mg, 0.1 mg, or 0.05 mg from the number given following the term.

In one specific embodiment, the sumatriptan formulation is at a concentration of 24 mg/ml comprising: 33.6 mg sumatriptan succinate; 0.71 mg dibasic sodium phosphate anhydrous; 19.49 mg mannitol; NaOH to adjust to pH 5.55 with a measured osmolality: 309 mmol/L. In another specific embodiment the sumatriptan formulation is at a concentration of 30 mg/ml comprising: 42.0 mg sumatriptan succinate; 0.71 mg dibasic sodium phosphate anhydrous; 12.21 mg mannitol; NaOH to adjust pH to 5.5; with a measured osmolality: 306 mmol/L.

By decreasing the total fluid volume of the injection and coupling this with microneedle delivery, several benefits are achieved using the methods of the invention including but not limited to a decrease mechanical pain perception due to tissue distention, and a reduction in mechanical pain perception due to the needle puncture. Decreased fluid volume also minimizes spillover of the ID injected solution to the subcutaneous tissue, and thereby maximizes the pharmacological benefits of ID delivery (specifically faster systemic onset).

In one embodiment, formulation of the invention contains a phosphate buffer with mannitol, dextrose, sorbitol, or any other sugar or carbohydrate based tonicity agent, in the absence of NaCl. Furthermore, without being limited by theory, by removing the salts resulting from tonicity agents such as NaCl, and employing sugar or carbohydrate based tonicity agent, formulation of the invention provides less skin irritation, and thus reduced pain, which can contribute to greater compliance. Suitable tonicity agents that may be used in connection with formulations of the invention include, but are not limited to, mannitol, dextrose, sorbitol, or any other sugar or carbohydrate based tonicity agent conventionally used in the art.

In yet another preferred embodiment, the invention provides a more concentrated sumatriptan formulation as described and exemplified herein coupled with microneedle administration so that the formulation is deposited in the intradermal compartment of a subjects' skin at a 0.5-3 mm depth range to provide the benefits disclosed herein. Although not intending to be bound by a particular mechanism of action the formulations of the invention, when administered in accordance with the methods of the invention, provide a faster uptake from the injection site, due in part to a controlled pH and reduced volume. In addition, the formulations of the invention, due in part to the absence of sodium chloride, causes less skin irritation and results in reduced pain. Furthermore, by pairing the formulations of the invention with an appropriate microneedle device such as Microinfusor for extended duration, or a microneedle based syringe or autoinjector, the timing of the injection can be specified to provide maximal comfort.

In contrast to previous parenteral injection formulation for SC administration, the invention provides improved formulations of sumatriptan succinate suitable for Intradermal and/or junctional delivery with improvements over conventional modes of delivery of sumatriptan succinate. The invention encompasses reformulated sumatriptan succinate formulations wherein the injection solution for ID administration has been modified to minimize the chemical and formulation effects responsible for nociception (pain perception) upon injection. In some embodiments, the invention provides sumatriptan succinate formulations wherein the solution has been buffered to a pH of 5.5 to be closer to the physiological pH range, and the sodium chloride excipients have been minimized to reduce the overall ionic strength of the solution, and reduce the levels of Na⁺ and Cl⁻ ions which may also be responsible for increased pain perception.

The formulations of the invention are particularly useful for the use of intradermal and/or junctional “metered bolus” infusions over a period of tens of seconds to minutes which among other benefits is also expected to decrease the overall patient perception associated with parenteral administration of sumatriptan. In addition, the faster uptake allowed by Intradermal and/or junctional injection will reduce residence time of the drug at the injection site and potentially reduce irritation caused by the drug itself from prolonged contact with the tissues.

The methods of the invention are particularly effective over traditional methods of delivery in that they are less painful; result in less skin irritation; have a shortened or equivalent onset time; result in higher bioavailability; result in the reduction of the injection volume; and have improved compliance when partnered with delivery devices utilizing novel intradermal and/or junctional delivery devices and microneedles.

The invention provides new sumatriptan formulations which are not detrimental to the skin and preferably have an advantage over the current formulation, Imitrex. Using the methods of the invention delivering sumatriptan succinate in accordance with the invention with a reduced fluid volume reduces the effects of erythema and edema in the skin. Using the methods of the invention, the smaller delivery volumes (up to 250 μl) are better suited for delivery through microneedles, targeting dermis and junctional space, taking full advantage of the enhanced PK effect. These microneedles cause less tissue trauma and are less painful than standard needles and may help improve patient compliance when “partnered” with a drug that is formulated for the intradermal and/or junctional route. Sumatriptan succinate can be reformulated to minimize skin effects, potentially maximize performance and improve patient compliance.

### 4. DESCRIPTION OF THE FIGURES

FIG. 1 Erythema: Solution by Time Interaction

FIG. 2. Edema Solution by time Interaction

FIG. 3 Edema: Depth by Time Interaction

FIG. 4 Main Effects Plot: Data means for erythema

FIG. 5 Main Effects Plot: Data Means for edema

FIG. 6 Interaction Plot: Data Means for Erythema

FIG. 7 Edema by Depth and Solution Over time: 12 mg/mL at 1.5 mm

FIG. 8 Edema by Depth and Solution Over time: 12 mg/mL at 2 mm

FIG. 9 Edema by Depth and Solution Over time: 12 mg/mL at 3 mm

FIG. 10 Edema by Depth and Solution Over time: 24 mg/mL at 1.5 mm

FIG. 11 Edema by Depth and Solution Over time: 24 mg/mL at 2 mm

FIG. 12 Edema by Depth and Solution Over time: 24 mg/mL at 3 mm

FIG. 13A Edema by Depth and Solution Over time: 30 mg/mL at 1.5 mm

FIG. 13B Edema by Depth and Solution Over time: 30 mg/ml at 2 mm

FIG. 14 Edema by Depth and Solution Over time: 30 mg/mL at 3 mm

FIG. 15 Erythema by Depth and Solution Over time: 12 mg/mL at 1.5 mm

FIG. 16 Erythema by Depth and Solution Over time: 12 mg/mL at 2 mm

FIG. 17 Erythema by Depth and Solution Over time: 12 mg/mL at 3 mm

FIG. 18 Erythema by Depth and Solution Over time: 24 mg/mL at 1.5 mm

FIG. 19 Erythema by Depth and Solution Over time: 24 mg/mL at 2 mm

FIG. 20 Erythema by Depth and Solution Over time: 24 mg/mL at 3 mm

FIG. 21 Erythema by Depth and Solution Over time: 30 mg/mL at 1.5 mm

FIG. 22 Erythema by Depth and Solution Over time: 30 mg/mL at 2 mm

FIG. 23 Erythema by Depth and Solution Over time: 30 mg/mL at 3 mm

FIG. 24 Average blood plasma levels for Yucatan mini-swine injected using a rapid ID bolus using syringe based ID needles. v SC

FIG. 25. Average blood plasma levels for Yucatan mini-swine injected using a metered bolus infusion ID using syringe based ID needles. v SC

FIG. 26 Average blood plasma levels for Yucatan mini-swine injected ID using syringe based ID needles. v SC

FIG. 27 Average blood plasma levels for Yucatan mini-swine injected using a metered bolus infusion ID using syringe based ID needles. v SC

FIG. 28 Average blood plasma levels for Yucatan mini-swine injected using a metered bolus infusion ID using syringe based ID needles. v SC

FIG. 29 Average blood plasma levels for Yucatan mini-swine injected using a metered bolus infusion ID using syringe based ID needles. v SC

FIG. 30 Average blood plasma levels for Yucatan mini-swine injected using a metered bolus infusion ID using syringe based ID needles. v SC

FIG. 30 Average blood plasma levels for Yucatan mini-swine injected using a metered bolus infusion ID using syringe based ID needles. v SC

FIG. 31 Average blood plasma levels for Yucatan mini-swine injected using a metered bolus infusion ID using syringe based ID needles. v SC

FIG. 32 Average blood plasma levels for Yucatan mini-swine injected using a metered bolus infusion ID using syringe based ID needles. v SC

FIG. 33 Average blood plasma levels for Yucatan mini-swine injected using a metered bolus infusion ID using syringe based ID needles. v SC

FIG. 34 Average blood plasma levels for Yucatan mini-swine injected using a metered bolus infusion ID using syringe based ID needles. v SC

FIG. 35 Average blood plasma levels for Yucatan mini-swine injected using a metered bolus infusion ID using syringe based ID needles. v SC

### 5. DETAILED DESCRIPTION OF THE INVENTION

The present invention provides a method for treatment and/or prevention, management and control of varying types and severities of pain and related syndromes, by administering an agent for management of pain, particularly anti-migraine agents, more particularly sumatriptan succinate, to the intradermal and/or junctional compartment of a subject's skin, preferably a human, using the methods and devices disclosed herein. In most preferred embodiments, the invention relates to the treatment, prevention and management of migraine and associated conditions, including but not limited to migraine without aura (“common migraine”), migraine with aura (“classic migraine”), migraine with typical aura, migraine with prolonged aura, familial hemiplegic migraine, basilar migraine, migraine aura without headache, migraine with acute-onset aura, opthalmoplegic migraine, retinal migraine, cluster headaches, chronic paroxysmal hemicrania, headache associated with vascular disorders, tension headache and pediatric migraine by intradermal and/or junctional delivery of agents for management of pain, particularly anti-migraine agents, more particularly sumatriptan succinate, to a subject, preferably humans, by directly targeting the dermal or junctional space, whereby such method alters the pharmacokinetics (PK) and pharmacodynamics (PD) parameters of the administered agent. Thus, the methods of the invention are particularly useful for the treatment, prevention and/or management of migraine and associated conditions. In some embodiments, the agent for management of pain, particularly anti-migraine agents, more particularly sumatriptan succinate is deposited to the upper region of the dermis (i.e., the dermal vasculature). Once the agent is infused according to the methods of the invention to the dermal vasculature it exhibits pharmacokinetics superior to, and more clinically desirable than that observed for such agents when administered by conventional methods, e.g., SC or IM injection or oral delivery.

Agents for management of pain including anti-migraine agents (e.g., sumatriptan succinate) delivered in accordance with the methods of the invention have an improved clinical utility and therapeutic efficacy relative to other delivery methods including subcutaneous, intraperitoneal, or intramuscular delivery. The present invention provides benefits and improvements over conventional delivery methods including but not limited to improved pharmacokinetics, enhanced half life of circulating agent, reduction of undesired and harmful side-effects, reduction in severity and recurrence of adverse events (e.g., injection site reactions, pain, redness, stinging, swelling, edema, erthema, etc.), enhanced patient comfort and compliance, and overall enhanced therapeutic efficacy.

While not intending to be bound by any theoretical mechanism of action, the rapid absorption observed upon administration into the dermal vasculature is achieved as a result of the rich plexuses of blood and lymphatic vessels therein. One possible explanation for the unexpected enhanced absorption reported herein is that upon injection of agents for management of pain including anti-migraine agents (e.g., sumatriptan succinate) so that it readily reaches the dermal vasculature, an increase in blood flow and capillary permeability results. For example, it is known that a pinprick insertion to a depth of 3 mm produces an increase in blood flow and this has been postulated to be independent of pain stimulus and due to tissue release of histamine (Arildsson et al., 2000 Microvascular Res. 59:122-130). This is consistent with the observation that an acute inflammatory response elicited in response to skin injury produces a transient increase in blood flow and capillary permeability (see, Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172, 1971). At the same time, the injection into the intradermal layer would be expected to increase interstitial pressure. It is known that increasing interstitial pressure from values (beyond the “normal range”) of about −7 to about +2 mm Hg distends lymphatic vessels and increases lymph flow (Skobe et al., 2000 J. Investig. Dermatol. Symp. Proc. 5:14-19). Thus, the increased interstitial pressure elicited by injection into the intradermal layer is believed to elicit increased lymph flow and increased absorption of substances injected into the dermis.

5.1 Administration Methods

The present invention encompasses methods delivery of therapeutically or prophylactically effective amounts of agents for management of pain, particularly anti-migraine agents, more particularly triptan compounds, to the intradermal and/or junctional compartment of a subject's skin, preferably by selectively and specifically targeting the intradermal and/or junctional compartment without passing through it. Preferred anti-migraine agents are triptan compounds. As used herein, “triptan compounds” refer to the group of chemical compounds that contain 2-(1H-indol-3-yl)-N,N-dimethylethanamine moiety. In accordance with this invention, the triptan compounds include, but are not limited to, almotriptan, zolmitriptan, rizatriptan, sumatriptan, naratriptan, or pharmaceutically acceptable salts thereof. Preferred salts are almotriptan malate, rizatriptan benzoate, sumatriptan succinate, and naratriptan hydrochloride. Most preferred compound is sumatriptan succinate. Although methods of the invention are described in connection with sumatriptan succinate by way of an example, the use of other triptan compounds are also encompassed and can be optimized based on the description using well-known methods in the art.

In a most preferred embodiment, the intradermal and/or junctional compartment is targeted directly. The formulations of the invention have an improved absorption uptake within the intradermal and/or junctional space as compared to conventional delivery routes.

The term “intradermal (ID) administration” of an agent, as used in connection with methods of the invention, means the agent is delivered to the skin compartment known as the dermis, which is located beneath the epidermis. The dermis includes the papillary dermis and the reticular dermis. Typically, intradermal administration involves depositing an agent into the skin at a depth of from about 0.5 mm to about 2 mm, preferably from about 1 mm to about 2 mm. The term “junctional administration” of an agent, as used in connection with methods of the invention, means that the agent is delivered to the interface skin compartment that separates the reticular dermis and subcutaneous tissue. Typically, junctional administration of an agent involves depositing the agent into the skin at a depth of from about 2 mm to about 3 mm, preferably from about 2.5 mm to about 3 mm. The term “about,” as used herein, is used to denote that the depths are approximate. Specifically, the term “about” encompasses deviations of less than 0.5 mm, 0.3 mm, 0.2 mm, 0.1 mm, or 0.05 mm from the number given following the term.

Methods of the invention offer improved delivery properties as compared to conventional delivery routes, in particular, SC. Sumatriptan is typically administered to what is conventionally identified as the SC compartment of the skin. Conventional delivery to the SC compartment requires delivery at a depth of at least 5 mm, ranging typically from 8 mm to 13 mm.

Once a formulation containing the agent to be delivered is prepared, the formulation is typically transferred to an injection device for intradermal and/or junctional compartment delivery, e.g., a syringe. Delivery of the formulations of the invention in accordance with the methods of the invention provides an improved therapeutic and clinical efficacy of the substance over conventional modes of delivery including oral, IM and SC by specifically and selectively, preferably directly targeting the intradermal and/or junctional compartment. The delivery methods of the invention provide benefits and improvements such as, but not limited to, improved pharmacokinetics, reduced immunogenicity, and reduction of undesired immune response. The methods of the present invention result in improved pharmacokinetics such as an improved absorption uptake within the intradermal and/or junctional compartment. The formulations of the invention may be delivered to the intradermal and/or junctional space as a bolus or by infusion.

The formulations of the invention may be administered using any of the devices and methods disclosed in U.S. patent application Ser. Nos. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The intradermal and/or junctional methods of administration comprise microneedle-based injection and infusion systems or any other means to accurately target the intradermal and/or junctional space. The methods of administration encompass not only microdevice-based injection means, but other delivery methods such as needle-less or needle-free ballistic injection of fluids or powders into the intradermal and/or junctional space, Mantoux-type injection, enhanced ionotophoresis through microdevices, and direct deposition of fluid, solids, or other dosing forms into the skin.

The formulations of the invention comprising therapeutically or prophylactically effective amounts of agents disclosed herein may be administered to intradermal and/or junctional compartment of a subject's skin using, for example, a Mantoux type injection, see, e.g., Flynn et al., 1994, Chest 106: 1463-5, which is incorporated herein by reference in its entirety. For example, the formulation of the invention may be delivered to the intradermal and/or junctional compartment of a subject's skin using the following exemplary method. The formulation is drawn up into a syringe, e.g., a 1 mL latex free syringe with a 20 gauge needle; after the syringe is loaded it is replaced with a 30 gauge needle for administration. The skin of the subject, e.g., mouse, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention encompasses the use of conventional injection needles, catheters or microneedles of all known types, employed singularly or in multiple needle arrays. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. The invention encompasses ballistic fluid injection devices, powder jet delivery devices, piezoelectric, electromotive, electromagnetic assisted delivery devices, gas-assisted delivery devices, which directly penetrate the skin to directly deliver the formulations of the invention to the targeted location within the dermal space.

The actual method by which the formulations comprising an agent of the invention are targeted to the intradermal and/or junctional space is not critical as long as it penetrates the skin of a subject to the desired targeted depth within the intradermal and/or junctional space without passing through it. The actual optimal penetration depth will vary depending on the thickness of the subject's skin. In most cases, skin is penetrated to a depth of about 0.5-3 mm. Regardless of the specific device and method of delivery, the methods of the invention preferably targets the formulations of the invention to a depth of at least about 0.5 mm up to a depth of no more than 3 mm, preferably from about 1 mm to about 3 mm, from about 1.5 mm to about 3 mm, or from about 2 mm to about 3 mm. In some embodiments, the formulations are delivered at a targeted depth just under the stratum corneum and encompassing the epidermis and upper dermis, e.g., about 0.025 mm to about 3 mm. Where targeting specific cells in the skin is desired, the preferred target depth depends on the particular cell being targeted and the thickness of the skin of the particular subject. For example, if targeting the Langerhan's cells in the dermal space of human skin is desired, then the delivery would need to encompass, at least, in part, the epidermal tissue depth typically ranging from about 0.025 mm to about 0.2 mm in humans.

The formulations comprising an agent of the invention is delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

The invention also encompasses varying the targeted depth of delivery of formulations of the invention. The targeted depth of delivery of formulations may be controlled manually by the practitioner, with or without the assistance of an indicator to indicate when the desired depth is reached. Preferably, however, the devices used in accordance with the invention have structural means for controlling skin penetration to the desired depth within the intradermal and/or junctional space. The targeted depth of delivery may be varied using any of the methods described in U.S. patent application Ser. Nos. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; 10/028,988, filed on Dec. 28, 2001; or International Publication Nos. EP 10922 444, published Apr. 18, 2001; WO 01/02178, published Jan. 10, 2002; and WO 02/02179, published Jan. 10, 2002; all of which are incorporated herein by reference in their entirety.

The above-mentioned PK and PD benefits are best realized by accurate direct targeting of the dermal or junctional space. This is accomplished, for example, by using microneedle systems of less than about 250 micron outer diameter, and less than 3 mm exposed length. Such systems can be constructed using known methods of various materials including steel, silicon, ceramic, and other metals, plastic, polymers, sugars, biological and/or biodegradable materials, and/or combinations thereof.

It has been found that certain features of the intradermal and/or junctional administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal and/or junctional space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion and to leak into the lower pressure regions of the skin, such as the subcutaneous tissue. That is, at a greater depth a needle outlet with a greater exposed height will still seal efficiently where as an outlet with the same exposed height will not seal efficiently when placed in a shallower depth within the intradermal and/or junctional space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of substances to the dermal or junctional space.

It has also been found that by controlling the pressure of injection or infusion the high backpressure exerted during Intradermal and/or junctional administration can be overcome. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin and/or into the subcutaneous region. The appropriate delivery rates and volumes to obtain these effects may be determined experimentally using only ordinary skill. Increased spacing between multiple needles allows broader fluid distribution and increased rates of delivery or larger fluid volumes. In addition, it has been found that Intradermal and/or junctional infusion or injection often produces higher initial plasma levels of sumatriptan than conventional SC administration. This may allow for smaller doses of sumatriptan to be administered via the ID route.

The formulations comprising an agent of the invention may be administered using any of the devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

Preferably the devices for administration in accordance with the methods of the invention have structural means for controlling skin penetration to the desired depth within the intradermal and/or junctional space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 3 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of agent delivered in a given period of time. The needle may eject its agent from the end, the side or both. Microneedles may be incorporated into a variety of devices such as holders and housings that may also serve to limit the depth of penetration. The dermal-access means of the invention may also incorporate reservoirs to contain the agent prior to delivery or pumps or other means for delivering the drug or other substance under pressure. Alternatively, the device housing the dermal-access means may be linked externally to such additional components.

The methods of administration comprise microneedle-based injection and infusion systems or any other means to accurately target the intradermal and/or junctional space. The methods of administration encompass not only microdevice-based injection means, but other delivery methods such as needle-less or needle-free ballistic injection of fluids or powders into the intradermal and/or junctional space, Mantoux-type injection, enhanced ionotophoresis through microdevices, and direct deposition of fluid, solids, or other dosing forms into the skin.

In some embodiments, the present invention provides a drug delivery device including a needle assembly for use in making intradermal and/or junctional injections. The needle assembly has an adapter that is attachable to prefillable containers such as syringes and the like. The needle assembly is supported by the adapter and has a hollow body with a forward end extending away from the adapter. A limiter surrounds the needle and extends away from the adapter toward the forward end of the needle. The limiter has a skin engaging surface that is adapted to be received against the skin of an animal such as a human. The needle forward end extends away from the skin engaging surface a selected distance such that the limiter limits the amount or depth that the needle is able to penetrate through the skin of a subject.

In a specific embodiment, the hypodermic needle assembly for use in the methods of the invention comprises the elements necessary to perform the present invention directed to an improved method of delivering formulations comprising an agent of the invention into the skin of a subject's skin, preferably a human subject's skin, comprising the steps of providing a drug delivery device including a needle cannula having a forward needle tip and the needle cannula being in fluid communication with a formulation contained in the drug delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the substance from the drug delivery device through the needle cannula tip into the skin of the animal.

In a preferred embodiment, the invention encompasses a self-administered intradermal device for use with sumatriptan succinate for the treatment of migraine headaches in humans. The optimal device will combine minimal dermal irritation to the subject, minimal pain upon injection, would incorporate device based convenience features, and provide maximal onset of pain relief from migraine headache.

5.2 Formulation of the Invention

The invention encompasses formulations comprising any agent known in the art or disclosed herein for the treatment, prevention, management and control of pain for use in accordance with the methods of the invention. In some embodiments, the formulations of the invention comprise a therapeutically or prophylactically effective amount of an agent known in the art or disclosed herein for the treatment, prevention, management and control of pain and one or more other additives. Preferred agents are anti-migraine agents. Preferred anti-migraine agents are triptan compounds. As used herein, “triptan compounds” refer to the group of chemical compounds that contain 2-(1H-indol-3-yl)-N,N-dimethylethanamine moiety. In accordance with this invention, the triptan compounds include, but are not limited to, almotriptan, zolmitriptan, rizatriptan, sumatriptan, naratriptan, or pharmaceutically acceptable salts thereof. Preferred salts are almotriptan malate, rizatriptan benzoate, sumatriptan succinate, and naratriptan hydrochloride. Most preferred compound is sumatriptan succinate.

Almotriptan is chemically named as 1-[[[3-[2-(dimethylamino)ethyl]-1H-indol-5-yl]methyl]sulfonylpyrolidine, and its malate salt is commercially available under the trade name Axert®. Zolimitriptan is chemically named as (S)-4-[[3-[2-(dimethylamino)ethyl]-1H-indol-5-yl]methyl]-2-oxazolidinone, and is commercially available under the trade name Zomig®. Rizatriptan is chemically named as N,N-dimethyl-5-(1H-1,2,4-triazol-1-ylmethyl)-1H-indole-3-ethanamine, and its monobenzoate salt is commercially available under the trade name Maxalt®. Sumatriptan is chemically named as 3-[2-(dimethylamino)ethyl]-N-methyl-indole-5-methanesulfonamide, and its succinate salt is available under the trade name Imitrex®. Naratriptan is chemically named as N-methyl-3-(1-methyl-4-piperidinyl)-1H-indole-5-ethanesulfonamide, and its hydrochloride salt is available under the trade name Amerge®.

Although the formulations of the invention are described in connection with sumatriptan succinate by way of an example, the use of other agents, in particular other triptan compounds, are also encompassed and can be optimized based on the description using well-known methods in the art.

Additives that may be used in the formulations of the invention include for example, wetting agents, emulsifying agents, or pH buffering agents. The formulations of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition), the entirety of which is incorporated herein by reference. These formulations may be sterilized by conventional sterilization techniques, or may be sterile filtered. The formulations may contain pharmaceutically acceptable auxiliary substances as required to approximate physiological conditions, such as pH buffering agents. Useful buffers include for example, sodium acetate/acetic acid buffers. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. In a preferred embodiment, sugar or carbohydrate-based tonicity agents such as, but not limited to, dextrose, mannitol, and sorbitol are used in formulations of the invention to reduce the skin irritation.

The agents for use in the methods of the invention can also be formulated as pharmaceutically acceptable salts (e.g., acid addition salts) and/or complexes thereof. Pharmaceutically acceptable salts are non-toxic salts at the concentration at which they are administered. Although not intending to be bound by a particular mechanism of action, the preparation of such salts can facilitate the pharmacological use by altering the physical-chemical characteristics of the formulation without preventing the formulation from exerting its physiological effect. Examples of useful alterations in physical properties include increasing the solubility to facilitate the administration of higher concentrations of the drug. Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Generally, carriers or excipients known in the art can also be used to facilitate administration of the formulations of the present invention. Examples of carriers and excipients include but are not limited to calcium carbonate, calcium phosphate, various sugars such as lactose, or types of starch, cellulose derivatives, gelatin, vegetable oils, polyethylene glycols and physiologically compatible solvents. If desired, solutions of the above dosage compositions may be thickened with a thickening agent such as methylcellulose. They may be prepared in emulsified form, such as either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

The agents for management of pain, particularly anti-migraine agents, more particularly sumatriptan succinate may be in any form suitable for intradermal and/or junctional delivery. In one embodiment, the formulation of the invention is in the form of a flowable, injectable medium, i.e., a low viscosity formulation that may be injected in a syringe. The flowable injectable medium may be a liquid. Alternatively, the flowable injectable medium is a liquid in which particulate material is suspended, such that the medium retains its fluidity to be injectable and syringable, e.g., can be administered in a syringe. In most preferred embodiments, the invention encompasses a formulation of sumatriptan succinate, which meets volumetric limitations for intradermal and/or junctional injection, has a concentration of sufficient strength to provide the recommended dosage of sumatriptan (6 mg), and is physiologically acceptable for intradermal and/or junctional administration (e.g., causes minimal dermal irritation at the injection site).

In one embodiment, the invention provides an improved formulation of injectable sumatriptan succinate to make it acceptable for delivery to the intradermal and/or junctional space. The improved formulation may also be delivered via conventional routes of delivery including, but not limited to, delivery to SC and IM compartments and oral delivery. Formulation of the invention contains sumatriptan succinate at a higher concentration than conventionally used formulations, including, but limited to, greater than about 20 mg/ml, about 24 mg/ml, or about 30 mg/ml. In some embodiments, formulation of the invention contains sumatriptan succinate at a concentration of from about 20 mg/ml to about 60 mg/ml, from about 20 mg/ml to about 40 mg/ml, from about 25 mg/ml to about 40 mg/ml, from about 20 mg/ml to about 30 mg/ml, from about 23 mg/ml to about 35 mg/ml, or from about 25 mg/ml to about 30 mg/ml.

In one specific embodiment, the sumatriptan formulation is at a concentration of 24 mg/ml comprising: 33.6 mg sumatriptan succinate; 0.71 mg dibasic sodium phosphate anhydrous; 19.49 mg mannitol; NaOH to adjust to pH 5.55 with a measured osmolality: 309 mmol/L. In another specific embodiment the sumatriptan formulation is at a concentration of 30 mg/ml comprising: 42.0 mg sumatriptan succinate; 0.71 mg dibasic sodium phosphate anhydrous; 12.21 mg mannitol; NaOH to adjust pH to 5.5; with a measured osmolality: 306 mmol/L.

In contrast to conventional parenteral injection formulation for SC administration, the invention provides improved formulations of sumatriptan succinate suitable for Intradermal and/or junctional delivery with improvements over conventional modes of delivery of sumatriptan succinate. The invention encompasses reformulated sumatriptan succinate formulations wherein the injection solution for ID administration has been modified to minimize the chemical and formulation effects responsible for nociception (pain perception) upon injection. In some embodiments, the invention provides sumatriptan succinate formulations wherein the solution has been buffered to a pH of 5.5 to be closer to the physiological pH range, and the sodium chloride excipients have been minimized to reduce the overall ionic strength of the solution, and reduce the levels of Na⁺ and Cl⁻ ions which may also be responsible for increased pain perception.

The agents for management of pain, particularly anti-migraine agents, more particularly sumatriptan succinate of the present invention can be prepared as unit dosage forms. A unit dosage per vial may contain 0.1 to 0.5 mL of the formulation. In some embodiments, a unit dosage form of the intradermal formulations of the invention may contain 50 μL to 100 μL, 50 μL to 200 μL, or 50 μL to 500 μL of the formulation. If necessary, these preparations can be adjusted to a desired concentration by adding a sterile diluent to each vial.

In yet other preferred embodiments, the invention provides a formulation of sumatriptan succinate for intradermal and/or junctional delivery of sumatriptan to enhance user acceptance of parenteral therapy by reducing the physiological and perception factors associated with injection and also provides pharmacological benefits including but not limited to reduced time to onset of systemic bioavailability and pharmacological action, e.g., reduced time to pain relief onset.

The invention provides improved methods for reformulating injectable sumatriptan succinate to make it acceptable for delivery to the intradermal and/or junctional space. Although not intending to be bound by a particular mechanism of action, it has been demonstrated that a more concentrated formulation in a phosphate buffer with mannitol, dextrose, sorbitol, or other sugar or carbohydrate based tonicity agent will allow sumatriptan succinate to take full advantage of the intradermal and/or junctional delivery route. By decreasing the total fluid volume of the injection and coupling this with microneedle delivery, several benefits are achieved using the methods of the invention. Benefits include, but are not limited to, a decrease in mechanical pain perception due to tissue distention, and a reduction in mechanical pain perception due to the needle puncture. Decreased fluid volume also minimizes spillover of the injected solution to the subcutaneous tissue, and thereby maximizes the pharmacological benefits of Intradermal and/or junctional delivery.

The formulations to be delivered in accordance with the methods of the invention include, but are not limited to, solutions thereof in pharmaceutically acceptable diluents or solvents, emulsions, suspensions, gels, particulates such as micro- and nanoparticles either suspended or dispersed, as well as in-situ forming vehicles of the same. The formulations of the invention may be in any form suitable for intradermal and/or junctional delivery. In one embodiment, the formulation of the invention is in the form of a flowable, injectable medium, i.e., a low viscosity formulation that may be injected in a syringe or insulin pen. The flowable injectable medium may be a liquid. Alternatively, the flowable injectable medium is a liquid in which particulate material is suspended, such that the medium retains its fluidity to be injectable and syringable, e.g., can be administered in a syringe.

The formulations of the present invention can be prepared as unit dosage forms. A unit dosage per vial may contain 0.1 to 0.5 mL of the formulation. In some embodiments, a unit dosage form of the formulations of the invention may contain 50 μL to 100 μL, 50 μL to 200 μL, or 50 μL to 500 μL of the formulation. If necessary, these preparations can be adjusted to a desired concentration by adding a sterile diluent to each vial. Preferably, formulations administered in accordance with the methods of the invention are not administered in volumes whereby the intradermal and/or junctional space might become overloaded leading to partitioning to one or more other compartments, such as the SC compartment.

5.3 Methods of use and Target Conditions

The present invention relates to improved treatment, prevention, control and management of varying types and severities of pain and related syndromes including, but not limited to, nociceptive pain, neuropathic pain, acute pain, chronic pain, nociceptive pain resulting from physical trauma (e.g., a cut or contusion of the skin; or a chemical or thermal burn), osteoarthritis, rheumatoid arthritis or tendonitis, myofascial pain, modifying mixed pain (i.e., pain with both nociceptive and neuropathic components), visceral pain; headache pain (e.g., migraine headache pain); mixed pain (i.e., chronic pain having nociceptive and neuropathic components); reflex neurovascular dystrophy; reflex dystrophy; sympathetically maintained pain syndrome; causalgia; Sudeck atrophy of bone; algoneurodystrophy; shoulder hand syndrome; post-traumatic dystrophy; autonomic dysfunction; cancer-related pain; phantom limb pain; fibromyalgia; myofascial pain; chronic fatigue syndrome; post-operative pain; spinal cord injury pain; central post-stroke pain; radiculopathy; sensitivity to temperature, light touch or color change to the skin (allodynia); pain from hyperthermic or hypothermic conditions; and other painful conditions (e.g., diabetic neuropathy, luetic neuropathy, postherpetic neuralgia, trigeminal neuralgia, or painful neuropathy induced iatrogenically by drugs such as vincristine, velcade or thalidomide).

In most preferred embodiments, the invention relates to the treatment, prevention and management of migraine and associated conditions including, but not limited to, migraine without aura (“common migraine”), migraine with aura (“classic migraine”), migraine with typical aura, migraine with prolonged aura, familial hemiplegic migraine, basilar migraine, migraine aura without headache, migraine with acute-onset aura, opthalmoplegic migraine, retinal migraine, cluster headaches, chronic paroxysmal hemicrania, headache associated with vascular disorders, tension headache and paediatric migraine by intradermal and/or junctional delivery of agents for management of pain, particularly anti-migraine agents, more particularly sumatriptan succinate, to a subject, preferably humans, by directly targeting the dermal or junctional space whereby such method alters the pharmacokinetic (PK) and pharmacodynamic (PD) parameters of the administered agent. Thus, the methods of the invention are particularly useful for the treatment, prevention and/or management of migraine and associated conditions.

Methods of this invention encompass methods for treating, preventing, managing and/or modifying various types of migraine, comprising administering a therapeutically or prophylactically effective amount of an agent for management of pain, particularly anti-migraine agents, more particularly sumatriptan succinate, to a patient in need thereof by delivering the agent to the intradermal and/or junctional compartment of the patient's skin using the methods and devices disclosed herein.

Methods of this invention encompass methods for treating, preventing, managing and/or modifying various types of pain and related syndromes, comprising administering a therapeutically or prophylactically effective amount of an agent for management of pain, particularly anti-migraine agents, more particularly sumatriptan succinate, to a patient in need thereof by delivering the agent to the intradermal and/or junctional compartment of the patient's skin using the methods and devices disclosed herein.

In one embodiment, the invention relates to a method for treating, preventing, managing and/or modifying nociceptive pain, comprising administering therapeutically or prophylactically effective amount of an agent for management of pain, particularly anti-migraine agents, more particularly sumatriptan succinate, to a patient in need thereof. In certain embodiments, the nociceptive pain results from physical trauma (e.g., a cut or contusion of the skin; or a chemical or thermal burn), osteoarthritis, rheumatoid arthritis or tendonitis. In another embodiment, the nociceptive pain is myofascial pain.

In another embodiment, the invention relates to a method for treating, preventing, managing and/or modifying neuropathic pain, comprising administering therapeutically or prophylactically effective amount of an agent for management of pain, particularly anti-migraine agents, more particularly sumatriptan succinate, to a patient in need thereof. In certain embodiments, the neuropathic pain is associated with stroke, diabetic neuropathy, luetic neuropathy, postherpetic neuralgia, trigeminal neuralgia, fibromyalgia, or painful neuropathy induced iatrogenically by drugs such as vincristine, velcade or thalidomide.

In another embodiment, the invention relates to a method for treating, preventing, managing and/or modifying mixed pain (i.e., pain with both nociceptive and neuropathic components), comprising administering therapeutically or prophylactically effective amount of an agent for management of pain, particularly anti-migraine agents, more particularly sumatriptan succinate, to a patient in need thereof by delivering the agent to the intradermal and/or junctional compartment of the patient's skin using the methods and devices disclosed herein.

In another embodiment, the invention relates to a method for treating, preventing, managing and/or modifying visceral pain; headache pain (e.g., migraine headache pain); mixed pain (i.e., chronic pain having nociceptive and neuropathic components); reflex neurovascular dystrophy; reflex dystrophy; sympathetically maintained pain syndrome; causalgia; Sudeck atrophy of bone; algoneurodystrophy; shoulder hand syndrome; post-traumatic dystrophy; autonomic dysfunction; cancer-related pain; phantom limb pain; fibromyalgia; myofascial pain; chronic fatigue syndrome; post-operative pain; spinal cord injury pain; central post-stroke pain; radiculopathy; sensitivity to temperature, light touch or color change to the skin (allodynia); pain from hyperthermic or hypothermic conditions; and other painful conditions (e.g., diabetic neuropathy, luetic neuropathy, postherpetic neuralgia, trigeminal neuralgia, or painful neuropathy induced iatrogenically by drugs such as vincristine, velcade or thalidomide), comprising administering a therapeutically or prophylactically effective amount of an agent for management of pain, particularly anti-migraine agents, more particularly sumatriptan succinate, to a patient in need thereof by delivering the agent to the intradermal and/or junctional compartment of the patient's skin using the methods and devices disclosed herein.

In a further embodiment, the invention relates to methods for treating a patient who has been previously treated for pain (in particular, a patient who was non-responsive to standard pain therapy), as well as a patient who has not previously been treated for pain, comprising administering an effective amount of a therapeutically or prophylactically effective amount of an agent for management of pain, particularly anti-migraine agents, more particularly sumatriptan succinate, to a patient in need thereof. Because a patient experiencing pain can have heterogeneous clinical manifestations and varying clinical outcomes, the treatment given to a patient can vary, depending on his/her prognosis. The skilled clinician will be able to readily determine without undue experimentation specific secondary agents, types of surgery, or types of physical therapy that can be effectively used to treat an individual patient.

In a yet a further embodiment, the invention relates to methods for managing the development and duration of pain, comprising administering to a patient in need of such management a therapeutically or prophylactically effective amount of an agent for management of pain, particularly anti-migraine agents, more particularly sumatriptan succinate, by delivering the agent to the intradermal and/or junctional compartment of the patient's skin using the methods and devices disclosed herein.

The invention further relates to methods for treating, preventing, managing and/or modifying pain, comprising administering therapeutically or prophylactically effective amount of an agent for management of pain, particularly anti-migraine agents, more particularly sumatriptan succinate, in combination with a second active agent, such as a prophylactic or therapeutic agent, to a patient in need thereof.

Examples of second active agents include, but are not limited to, conventional therapeutics used to treat, prevent, manage and/or modify pain, including, but not limited to, antidepressants, anticonvulsants, antihypertensives, anxiolytics, calcium channel blockers, muscle relaxants, non-narcotic analgesics, opioid analgesics, anti-inflammatories, cox-2 inhibitors, alpha-adrenergic receptor agonists or antagonists, ketamine, anesthetics, immunomodulatory agents, immunosuppressive agents, corticosteroids, hyperbaric oxygen, anticonvulsants, NMDA antagonists, IMiDs® and SelCIDs® (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference), or a combination thereof, and other therapeutics found, for example, in the μPhysician 's Desk Reference 2004.

The specific amount of the second active agent will depend on the specific agent used, the type of pain being treated or managed, the severity and stage of pain, and the amount(s) of the first agent for management of pain and any optional additional active agents concurrently administered to the patient. In a particular embodiment, the second active agent is salicyclic acid acetate, celocoxib, enbrel, thalidomide, an IMiD®, a SelCID®, gabapentin, phenytoin, carbamazepine, valproic acid, morphine sulfate, hydromorphone, prednisone, griseofulvin, penthonium, alendronate, dyphenhydramide, guanethidine, ketorolac, thyrocalcitonin, dimethylsulfoxide, clonidine, bretylium, ketanserin, reserpine, droperidol, atropine, phentolamine, bupivacaine, lidocaine, acetaminophen, nortriptyline, amitriptyline, imipramine, doxepin, clomipramine, fluoxetine, sertraline, nefazodone, venlafaxine, trazodone, bupropion, mexiletine, nifedipine, propranolol, tramadol, lamotrigine, ziconotide, ketamine, dextromethorphan, benzodiazepines, baclofen, tizanidine, phenoxybenzamine or a combination thereof, or a pharmaceutically acceptable salt, solvate, hydrate, stereoisomer, clathrate, prodrug or pharmacologically active metabolite thereof.

The invention further encompasses use of non-narcotic analgesics and anti-inflammatories to treat patients suffering from mild to moderate pain in combination with the methods of the invention. Anti-inflammatories such as non-steroidal anti-inflammatory drugs (NSAIDs) and cox-2 inhibitors typically inhibit inflammatory reactions and pain by decreasing activity of cyclo-oxygenase, which is responsible for prostaglandin synthesis. NSAIDs may provide pain relief in the early stage of a pain syndrome. Examples of anti-inflammatories include, but are not limited to, salicyclic acid acetate, ibuprofen, ketoprofen, rofecoxib, naproxen sodium, ketorolac, and other known conventional medications. Ibuprofen can be orally administered in an amount of 400-800 mg three times a day. See, e.g., Physicians' Desk Reference, 511, 667 and 773 (56^{th} ed., 2002); Physicians' Desk Reference for Nonprescription Drugs and Dietary Supplements, 511, 667, 773 (23^{rd} ed., 2002). Naproxen sodium may also preferably be used for relief of mild to moderate pain in an amount of about 275 mg thrice a day or about 550 mg twice a day. See, e.g., Physicians' Desk Reference, 2967-2970 (56^{th} ed., 2002). A specific cox-2 inhibitor is celocoxib.

Antidepressants, e.g., nortriptyline, may also be used in embodiments of the invention to treat patients suffering from chronic and/or neuropathic pain. Antidepressants increase the synaptic concentration of serotonin and/or norepinephrine in the CNS by inhibiting their reuptake by presynaptic neuronal membrane. Some antidepressants also have sodium channel blocking ability to reduce the firing rate of injured peripheral afferent fibers. Examples of antidepressants include, but are not limited to, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®) and other known conventional medications. See, e.g., Physicians' Desk Reference, 329, 1417, 1831 and 3270 (57^{th} ed., 2003). The oral adult dose is typically in an amount of about 25-100 mg, and preferably does not exceed 200 mg/d. A typical pediatric dose is about 0.1 mg/kg PO as initial dose, increasing, as tolerated, up to about 0.5-2 mg/d. Amitriptyline is preferably used for neuropathic pain in an adult dose of about 25-100 mg PO. See, e.g., Physicians' Desk Reference, 755, 1238, 1684 and 3495 (56^{th} ed., 2002).

Anticonvulsant drugs may also be used in embodiments of the invention. Examples of anticonvulsants include, but are not limited to, carbamazepine, oxcarbazepine (Trileptal®), gabapentin (Neurontin®), phenytoin, sodium valproate, clonazepam, topiramate, lamotrigine, zonisamide, and tiagabine. See, e.g., Physicians' Desk Reference, 2563 (57^{th} ed., 2003).

Another embodiment of the invention encompasses the use of narcotic analgesics. Examples of narcotic analgesics include, but are not limited to, morphine, heroin, hydromorphone, oxymorphone, levophanol, levallorphan, codeine, hydrocodone, oxycodone, nalmefene, nalorphine, naloxone, naltrexone, buprenorphine, butorphanol, nalbuphine, meperidine, diphenoxylate, loperamide, fentanyl, sufentanil, alfentanil, remifentanil, methadone, levomethadyl acetate, propoxyphene, pentazocine, dextromethorphane, levoproxyphene napsylate, noscapine, carbetapentane, caramiphene, chlophedianol, diphenhydramine, glaucine, phocodine, benzonatate, or other narcotic analgesics disclosed in, for example, Goodman & Gilman 's The Pharmacological Basis of Therapeutics, 10^{th} Ed, McGraw-Hill, pp. 569-619 (2001), which is incorporated herein by reference.

The invention encompasses any agents known in the art for the therapy of migraine headaches including, but not limited to, 5HT-1 (serotonin) receptor agonist class of medications, also known as the triptans, butalbital-containing products, and the ergot alkaloid products (e.g., ergotamine, dihydroergotamine, bromocriptine, ergonovine, methysergide). These agents are now considered first-line therapy for all types of migraines. Triptan products that may be used in accordance with the methods of the invention include, but are not limited to, Imitrex® (sumatriptan); Amerge® (naratriptan); Axert® (almotriptan); Maxalt® (rizatriptan); Zomig® (zolmitriptan); Frova® (frovatriptan); and Relpax® (eletriptan). The invention encompasses any agent known in the art for the acute treatment of mild or moderate migraine including but not limited to Aspirin, acetaminophen, ibuprofen, indomethacin, naproxen sodium, and isomethepten. The invention further encompasses any agent known in the art for the prophylactic treatment of severe migraine including, but not limited to, tricyclic antidepressant (e.g., amitriptylin, nortriptylin), sterotonergic antagonists (e.g., methysergide, cyproheptadine), B-adrenergic antagonists (e.g., propanaolol, timolol, atenolol, nadolol, metoprolol), and monoamine oxidase inhibitors (e.g., phenelzine, isocarboxazid).

In still another embodiment, this invention encompasses a method of treating, preventing, modifying, and/or managing pain, which comprises administering an agent for the management of pain in conjunction with physical therapy or psychological therapy.

Symptoms of pain include vasomotor dysfunction and movement disorders. A steady progression of gentle weight bearing to progressive active weight bearing is important in patients experiencing pain. Gradual desensitization to increasing sensory stimuli may also be helpful. Gradual increase in normalized sensation tends to reset the altered processing in the CNS. Physical therapy can thus play an important role in functional restoration. The goal of physical therapy is to gradually increase strength and flexibility.

In still another embodiment, this invention encompasses a method of treating, preventing, modifying, and/or managing pain, which comprises administering an agent disclosed herein in conjunction with (e.g., before, during, or after) pain management interventional techniques. Examples of pain management interventional techniques include, but are not limited to, the use of sympathetic blocks, intravenous regional blocks, placement of dorsal column stimulators or placement of intrathecal infusion devices for analgesic medication delivery. Preferred pain management interventional techniques provides a selective neural blockade which interrupts the activity of the sympathetic nervous system in the region in which pain is experienced.

### 6. EXAMPLES

6.1 Optimization of Sumatriptan Formulation

The objective of this study was to optimize the formulation of sumatriptan succinate, altering both concentration and volume, for use for intradermal and/or junctional delivery using the BD Micromedica drug delivery system.

The purpose of this randomized study was to investigate the skin effects of sumatriptan succinate at three concentrations when delivered intradermally or junctionally using microneedle drug delivery systems. One of the primary concerns of delivery of chemical compounds via the intradermal and/or junctional route is tissue damage or altered pathology in the dermis and epidermis due to the biochemical effects of these selected compounds. The commercially available solution, Imitrex (GlaxoWelcome) is available as a 12 mg/ml solution with a standard dose of 6 mg (0.5 mL) given subcutaneously. Skin effects at the injection site are a documented adverse effect in the package insert. This study investigated the skin effects of sumatriptan at two additional concentrations, 24 and 30 mg/ml solutions and their respective volumes, 250 and 200 μl delivered to the dermal or junctional space using BD Micromedica 30 and 34 gauge single needle devices with needle lengths of 1.5, 2 and 3 mm. Skin effects following the injection of sumatriptan at three concentrations were observed.

TABLE 1
CONDITIONS:
CONDITIONS

| | | | | | | |
|---|---|---|---|---|---|---|
| Sumatriptan | 12 | mg/ml | 24 | mg/ml | 30 | mg/ml |
| Concentrations | | | | | | |
| Needle Lengths | 1.5 | mm | 2.0 | mm | 3.0 | mm |
| Device Design/ | 30 | Ga | 34 | Ga | | |
| Gauge | | | | | | |

EXPERIMENTAL DESIGN: This study was a 3×3×2 fractional fractorial incomplete block design (See Table 1). A total of 12 Yorkshire Swine (Archer Farms) were used. Each pig received one injection according to a randomization schedule (RS) once a day for a total of nine injections. Animals were not exposed to all possible injection combinations, because of the incomplete block design.

A subset of the subject population are known to exhibit heightened dermal responses to sumatriptan injection. This effect was anticipated and the randomization schedule was prepared to account for this possibility. To minimize the responder/non-responder effects on the statistical outcomes of the study, pigs were screened prior to the study start date by receiving a single 0.5 ml SC injection of 12 mg/ml sumatriptan via the Imitrex STATdose system. These skin sites were observed immediately upon removal of the device and at 30 minutes, 1, 2, 3, 4, 6, 8 and 24 hours and skin effects documented using the Draize Dermal Irritation method. Pigs that had an observable Draize score of level 2 or above at time points following the first observation were anticipated to be responders. Responder pigs were assigned to the following pig numbers on the randomization table in the following order: 1, 4, 2, 5, 3, and 6.

SITE SELECTION: Injections 1-9 were given on alternating right and left flank, using the following format.

MATERIALS AND METHODS: Sumatriptan was at a concentration of 12 mg/ml Imitrex (GSK-0.6 mg/0.5 mL) Lot # C082699. Additionally 24 mg/ml of sumatriptan solution was prepared containing the following: 33.6 mg sumatriptan succinate; 0.71 mg dibasic sodium phosphate anhydrous; 19.49 mg mannitol; NaOH to adjust pH; pH5.55; 309 mmol/L.

30 mg/ml of sumatriptan solution was prepared containing the following: 42.0 mg sumatriptan succinate; 0.71 mg dibasic sodium phosphate anhydrous; 12.21 mg mannitol; NaOH to adjust pH; pH 5.50; 306 mmol/L.

DEVICES: The following devices were used.

Syringe based microneedle systems: 30 gauge 1.5 mm ID needle with skin penetration limiter (Lot #E216801); 2 mm ID needle with limiter incorporating a 30 gauge ½ inch length needle with an ID bevel and a skin penetration limiter to allow 2 mm penetration; 3 mm needle with limiter incorporating a 30 gauge ½ inch length needle with a bevel and a skin penetration limiter to allow 3 mm penetration. All syringe based devices were connected to an accurate leur lock (LL) volumetric syringe for measuring the dose volume, and were administered in a bolus injection fashion using manual control of delivery rate.

Catheter based microneedle systems: All catheter based microneedle systems consisted of a linear array of three 34 Ga microneedles with exposed microneedle lengths of 1.5, 2, or 3 mm respectively, which were mounted in an acrylic hub designed to insert the needles perpendicular to the skin surface. During the delivery period, the microneedle array is held in place flat against the skin via an integral adhesive ring incorporated on the hub. The catheter hub is also connected via an integral length of medical grade tubing to a Leur inlet, which is in turn connected to a 1 ml syringe as the drug reservoir. The flow rate and delivery volume from the syringe are controlled via a programmable, highly accurate volumetric syringe pump. Identification and lot numbers of the catheter devices used for this study were: 1.5-DA677 (Lot 5); 2-DA677 (Lot 5); and 3-DA677 (Lot 3).

EXPERIMENTAL DESIGN: All injections were performed under anesthesia. Pigs were fasted for 12-18 hours prior to anesthesia. A mixture of Rompun® (2 mg/kg), Telazol® (4 mg/kg), and Ketamine (2 mg/kg) were given IM to sedate. Atropine (0.02 mg/kg) was given IM directly after sedation to avoid excessive salivation. Swine were masked down with Isoflurane if needed during the injection time.

Animal Preparation:

TREATMENT DAY 1: The hair on both flanks of the pig were clipped and the skin wiped clean with chlorohexaderm scrub and alcohol. A clean, unblemished area was selected. Injection was performed according to a randomization schedule.

For 34-gauge catheter set devices, a BD 1 mL LL syringe and a Harvard syringe pump were used to control flow rate (100 μL/min). Devices remained in place for 1 minute following the injection. For 30-gauge syringe system, injections were performed over 50-60 seconds using manual rate control. After injection, photos were taken of skin effects. The skin site was marked with a permanent skin marker for later identification of site. Pigs were recovered in their runs.

TREATMENT DAY 2-9: All steps from Day 1 were repeated on alternate flanks of each pig following a pre-assigned randomization schedule.

OBSERVATIONS: Skin effects were observed using the Draize Dermal Irritation Scoring Method at the following times: Immediately upon removal of device from skin, 30 min, 1, 2, 3, 4, 6, 8 and 24 hours after injection

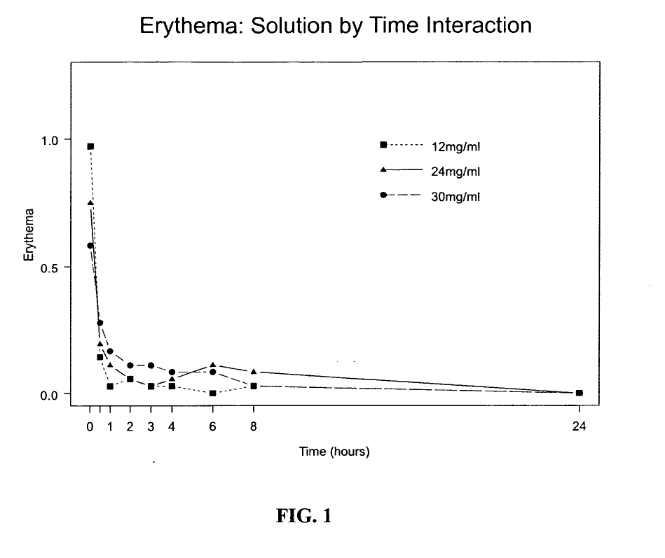

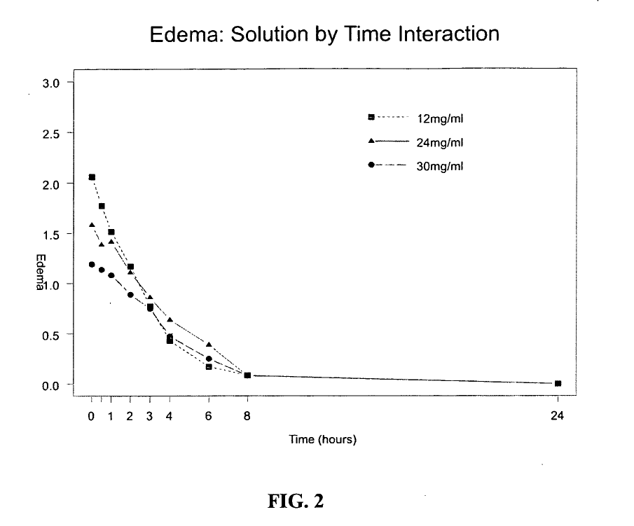

SOLUTION BY TIME INTERACTION: FIGS. 1 and 2 indicate that fluid volume has direct influence on Draize scores and irritation due to tissue distention. Higher erythema and edema scores were observed with the current formulation at time 0 than with the optimized formulations of lesser volumes. The 24 mg/ml and the 30 mg/ml formulations showed no additional deleterious effects in the skin indicating the higher concentrations and their respective volumes (250 and 200 μl) may even be beneficial in limiting erythema and edema. At the 30-minutes observation, erythema scores dropped dramatically for all formulations with Draize scores less than 1 (barely perceptible) to 0 (no erythema). All skin effects, edema and eythema were gone by the eight-hour observation for all three formulations.

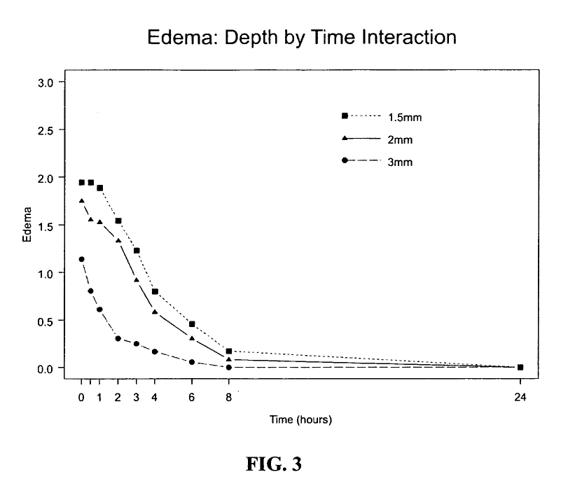

DEPTH BY TIME INTERACTION: The higher edema scores at times 0-8 hours are coupled with devices targeting the shallower dermis. Needle lengths of 1.5 mm and 2 mm delivered the fluid to the shallower regions of the dermis, and the edema was more evident. The 3 mm device delivered to the deeper region of the dermis and to junctional space, resulting in the infiltrate being less observable. Edema appears to resolve at the same rate for the 1.5 and 2 mm devices. The edema for the 3 mm devices appeared to resolve a little quicker (FIG. 3)

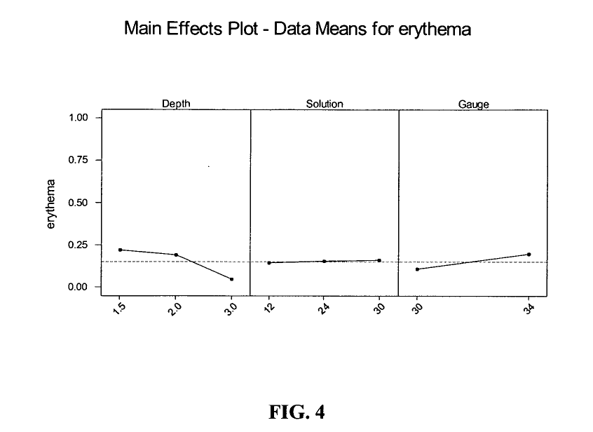

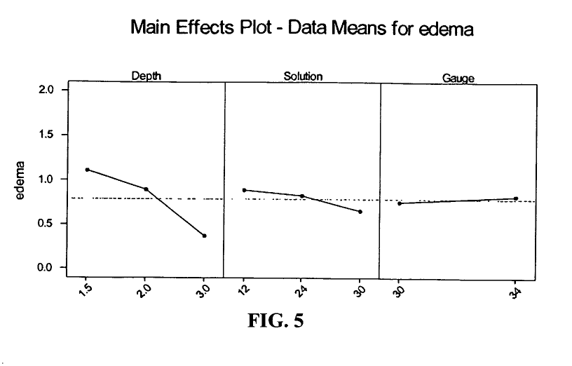

The Main Effects Plots below for erythema and edema indicate that, while depth and device configuration has an effect on erythema scores, the solution does not (FIGS. 4-5). There is no difference between the current formulation and the two new formulations when measured by Draize scoring for irritation. This indicates that the higher concentrations of sumatriptan are not detrimental to the skin. For edema, there is an effect for depth and solution. The higher edema scores reflect the actual fluid being instilled into the skin and the respective volumes of each formulation. It is clear that the higher fluid volume coupled with delivery to a shallow depth would be more visible on the skin surface producing higher edema scores than fluid delivered to a deeper region of the dermis.

STATISTICAL REPORT: Erythema and Edema scores were recorded at 9 time points: Initial ½h., 2h., 3h., 4h., 6h., 8h. and 24h. Table 2 presents summary statistics per Factor level (main effects averaged over all levels of the other factors).

TABLE 2
SUMMARY OF STATS

| | | | Edema | | | | | | Erythema | | | | | |
|---|---|---|---|---|---|---|---|---|---|---|---|---|---|---|
| Factor | Levels | Time | Mean | Median | SD | min | max | n | Mean | Median | SD | min | max | n |
| Gauge | 30 | 0 | 1.6 | 2 | 0.7 | 0 | 3 | 53 | 0.5 | 0.7 | 0 | 0 | 2 | 53 |
| | | 0.5 | 1.4 | 1 | 0.8 | 0 | 3 | 53 | 0.2 | 0.4 | 0 | 0 | 1 | 53 |
| | | 1 | 1.2 | 1 | 0.9 | 0 | 3 | 53 | 0.1 | 0.2 | 0 | 0 | 1 | 53 |
| | | 2 | 1.0 | 1 | 0.8 | 0 | 2 | 53 | 0.0 | 0.2 | 0 | 0 | 1 | 53 |
| | | 3 | 0.8 | 1 | 0.8 | 0 | 2 | 53 | 0.0 | 0.1 | 0 | 0 | 1 | 53 |
| | | 4 | 0.4 | 0 | 0.6 | 0 | 2 | 53 | 0.1 | 0.2 | 0 | 0 | 1 | 53 |
| | | 6 | 0.2 | 0 | 0.4 | 0 | 1 | 53 | 0.1 | 0.2 | 0 | 0 | 1 | 53 |
| | | 8 | 0.1 | 0 | 0.2 | 0 | 1 | 53 | 0.1 | 0.2 | 0 | 0 | 1 | 53 |
| | | 24 | 0.0 | 0 | 0.0 | 0 | 0 | 53 | 0.0 | 0.0 | 0 | 0 | 0 | 53 |
| | 34 | 0 | 1.6 | 2 | 0.7 | 0 | 3 | 54 | 1.0 | 0.9 | 1 | 0 | 3 | 54 |
| | | 0.5 | 1.5 | 2 | 0.8 | 0 | 3 | 54 | 0.3 | 0.6 | 0 | 0 | 3 | 54 |
| | | 1 | 1.4 | 2 | 0.9 | 0 | 3 | 54 | 0.1 | 0.4 | 0 | 0 | 2 | 54 |
| | | 2 | 1.1 | 1 | 0.9 | 0 | 3 | 54 | 0.1 | 0.3 | 0 | 0 | 1 | 54 |
| | | 3 | 0.8 | 1 | 0.8 | 0 | 2 | 54 | 0.1 | 0.3 | 0 | 0 | 1 | 54 |
| | | 4 | 0.6 | 0.5 | 0.7 | 0 | 2 | 54 | 0.1 | 0.2 | 0 | 0 | 1 | 54 |
| | | 6 | 0.3 | 0 | 0.5 | 0 | 1 | 54 | 0.1 | 0.3 | 0 | 0 | 1 | 54 |
| | | 8 | 0.1 | 0 | 0.3 | 0 | 1 | 54 | 0.0 | 0.2 | 0 | 0 | 1 | 54 |
| | | 24 | 0.0 | 0 | 0.0 | 0 | 0 | 54 | 0.0 | 0.0 | 0 | 0 | 0 | 54 |
| Depth | 1.5 | 0 | 1.9 | 2 | 0.6 | 1 | 3 | 35 | 1.2 | 0.8 | 1 | 0 | 3 | 35 |
| | | 0.5 | 1.9 | 2 | 0.6 | 1 | 3 | 35 | 0.3 | 0.4 | 0 | 0 | 1 | 35 |
| | | 1 | 1.9 | 2 | 0.6 | 1 | 3 | 35 | 0.1 | 0.4 | 0 | 0 | 1 | 35 |
| | | 2 | 1.5 | 2 | 0.6 | 0 | 3 | 35 | 0.1 | 0.3 | 0 | 0 | 1 | 35 |
| | | 3 | 1.2 | 1 | 0.7 | 0 | 2 | 35 | 0.1 | 0.3 | 0 | 0 | 1 | 35 |
| | | 4 | 0.8 | 1 | 0.5 | 0 | 2 | 35 | 0.1 | 0.2 | 0 | 0 | 1 | 35 |
| | | 6 | 0.5 | 0 | 0.5 | 0 | 1 | 35 | 0.1 | 0.3 | 0 | 0 | 1 | 35 |
| | | 8 | 0.2 | 0 | 0.4 | 0 | 1 | 35 | 0.0 | 0.2 | 0 | 0 | 1 | 35 |
| | | 24 | 0.0 | 0 | 0.0 | 0 | 0 | 35 | 0.0 | 0.0 | 0 | 0 | 0 | 35 |
| | 2.0 | 0 | 1.8 | 2 | 0.6 | 1 | 3 | 36 | 0.8 | 0.8 | 1 | 0 | 3 | 36 |
| | | 0.5 | 1.6 | 2 | 0.7 | 0 | 3 | 36 | 0.3 | 0.8 | 0 | 0 | 3 | 36 |
| | | 1 | 1.5 | 2 | 0.7 | 0 | 3 | 36 | 0.1 | 0.4 | 0 | 0 | 2 | 36 |
| | | 2 | 1.3 | 1 | 0.9 | 0 | 3 | 36 | 0.1 | 0.3 | 0 | 0 | 1 | 36 |
| | | 3 | 0.9 | 1 | 0.7 | 0 | 2 | 36 | 0.1 | 0.2 | 0 | 0 | 1 | 36 |
| | | 4 | 0.6 | 0 | 0.7 | 0 | 2 | 36 | 0.1 | 0.2 | 0 | 0 | 1 | 36 |
| | | 6 | 0.3 | 0 | 0.5 | 0 | 1 | 36 | 0.1 | 0.3 | 0 | 0 | 1 | 36 |
| | | 8 | 0.1 | 0 | 0.3 | 0 | 1 | 36 | 0.1 | 0.3 | 0 | 0 | 1 | 36 |
| | | 24 | 0.0 | 0 | 0.0 | 0 | 0 | 36 | 0.0 | 0.0 | 0 | 0 | 0 | 36 |
| | 3.0 | 0 | 1.1 | 1 | 0.7 | 0 | 2 | 36 | 0.3 | 0.5 | 0 | 0 | 2 | 36 |
| | | 0.5 | 0.8 | 1 | 0.7 | 0 | 3 | 36 | 0.0 | 0.2 | 0 | 0 | 1 | 36 |
| | | 1 | 0.6 | 0 | 0.7 | 0 | 2 | 36 | 0.0 | 0.2 | 0 | 0 | 1 | 36 |
| | | 2 | 0.3 | 0 | 0.6 | 0 | 2 | 36 | 0.0 | 0.2 | 0 | 0 | 1 | 36 |
| | | 3 | 0.3 | 0 | 0.6 | 0 | 2 | 36 | 0.0 | 0.2 | 0 | 0 | 1 | 36 |
| | | 4 | 0.2 | 0 | 0.4 | 0 | 1 | 36 | 0.1 | 0.2 | 0 | 0 | 1 | 36 |
| | | 6 | 0.1 | 0 | 0.2 | 0 | 1 | 36 | 0.0 | 0.0 | 0 | 0 | 0 | 36 |
| | | 8 | 0.0 | 0 | 0.0 | 0 | 0 | 36 | 0.0 | 0.0 | 0 | 0 | 0 | 36 |
| | | 24 | 0.0 | 0 | 0.0 | 0 | 0 | 36 | 0.0 | 0.0 | 0 | 0 | 0 | 36 |
| Solution | 12 | 0 | 2.1 | 2 | 0.7 | 0 | 3 | 35 | 1.0 | 0.8 | 1 | 0 | 2 | 35 |
| | | 0.5 | 1.8 | 2 | 0.9 | 0 | 3 | 35 | 0.1 | 0.4 | 0 | 0 | 1 | 35 |
| | | 1 | 1.5 | 2 | 0.9 | 0 | 3 | 35 | 0.0 | 0.2 | 0 | 0 | 1 | 35 |
| | | 2 | 1.2 | 1 | 1.0 | 0 | 3 | 35 | 0.1 | 0.2 | 0 | 0 | 1 | 35 |
| | | 3 | 0.8 | 1 | 0.8 | 0 | 2 | 35 | 0.0 | 0.2 | 0 | 0 | 1 | 35 |
| | | 4 | 0.4 | 0 | 0.6 | 0 | 2 | 35 | 0.0 | 0.2 | 0 | 0 | 1 | 35 |
| | | 6 | 0.2 | 0 | 0.4 | 0 | 1 | 35 | 0.0 | 0.0 | 0 | 0 | 0 | 35 |
| | | 8 | 0.1 | 0 | 0.3 | 0 | 1 | 35 | 0.0 | 0.2 | 0 | 0 | 1 | 35 |
| | | 24 | 0.0 | 0 | 0.0 | 0 | 0 | 35 | 0.0 | 0.0 | 0 | 0 | 0 | 35 |
| | 24 | 0 | 1.6 | 2 | 0.6 | 0 | 2 | 36 | 0.8 | 0.9 | 1 | 0 | 3 | 36 |
| | | 0.5 | 1.4 | 1.5 | 0.8 | 0 | 3 | 36 | 0.2 | 0.6 | 0 | 0 | 3 | 36 |
| | | 1 | 1.4 | 2 | 0.8 | 0 | 3 | 36 | 0.1 | 0.4 | 0 | 0 | 2 | 36 |
| | | 2 | 1.1 | 1 | 0.9 | 0 | 3 | 36 | 0.1 | 0.2 | 0 | 0 | 1 | 36 |
| | | 3 | 0.9 | 1 | 0.8 | 0 | 2 | 36 | 0.0 | 0.2 | 0 | 0 | 1 | 36 |
| | | 4 | 0.6 | 0.5 | 0.7 | 0 | 2 | 36 | 0.1 | 0.2 | 0 | 0 | 1 | 36 |
| | | 6 | 0.4 | 0 | 0.5 | 0 | 1 | 36 | 0.1 | 0.3 | 0 | 0 | 1 | 36 |
| | | 8 | 0.1 | 0 | 0.3 | 0 | 1 | 36 | 0.1 | 0.3 | 0 | 0 | 1 | 36 |
| | | 24 | 0.0 | 0 | 0.0 | 0 | 0 | 36 | 0.0 | 0.0 | 0 | 0 | 0 | 36 |
| | 30 | 0 | 1.2 | 1 | 0.6 | 0 | 2 | 36 | 0.6 | 0.8 | 0 | 0 | 3 | 36 |
| | | 0.5 | 1.1 | 1 | 0.8 | 0 | 2 | 36 | 0.3 | 0.6 | 0 | 0 | 3 | 36 |
| | | 1 | 1.1 | 1 | 0.8 | 0 | 2 | 36 | 0.2 | 0.4 | 0 | 0 | 1 | 36 |
| | | 2 | 0.9 | 1 | 0.8 | 0 | 2 | 36 | 0.1 | 0.3 | 0 | 0 | 1 | 36 |
| | | 3 | 0.8 | 1 | 0.8 | 0 | 2 | 36 | 0.1 | 0.3 | 0 | 0 | 1 | 36 |
| | | 4 | 0.5 | 0 | 0.6 | 0 | 2 | 36 | 0.1 | 0.3 | 0 | 0 | 1 | 36 |
| | | 6 | 0.3 | 0 | 0.4 | 0 | 1 | 36 | 0.1 | 0.3 | 0 | 0 | 1 | 36 |
| | | 8 | 0.1 | 0 | 0.3 | 0 | 1 | 36 | 0.0 | 0.2 | 0 | 0 | 1 | 36 |
| | | 24 | 0.0 | 0 | 0.0 | 0 | 0 | 36 | 0.0 | 0.0 | 0 | 0 | 0 | 36 |

STATISTICAL ANALYSIS: An ordinal logistic regression was used to determine which of the three factors and interactions had a significant effect on Edema and Erythema. The model included a pig effect, time effect, gauge, depth and solution main effects as well as all two-way interactions. The significant main effects and interactions were as follows:

Edema:

Time
Depth
Pig*Depth
Pig
Depth*Time
Solution
Solution*Time
   Erythema:
Time
Depth
Pig
Pig*Time
Gauge
Solution*Time
Pig*Solution
Gauge*Solution

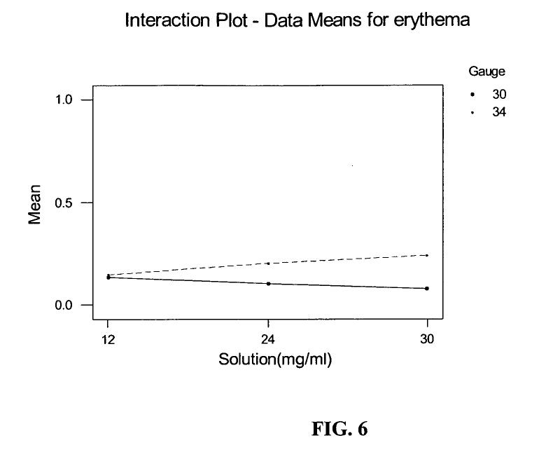

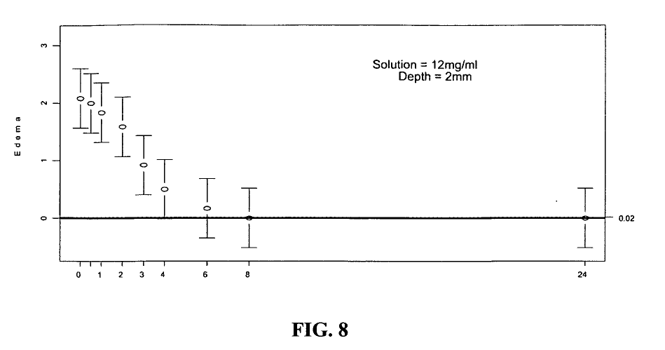

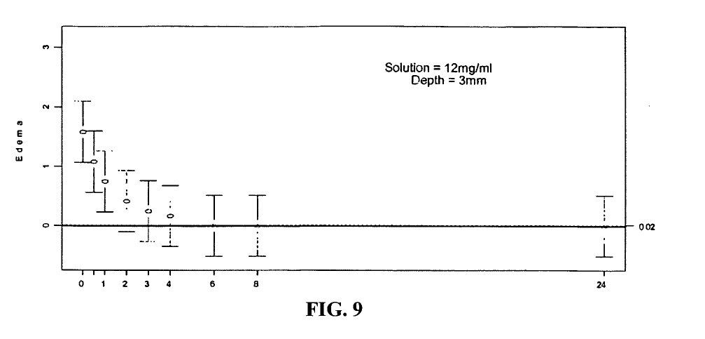

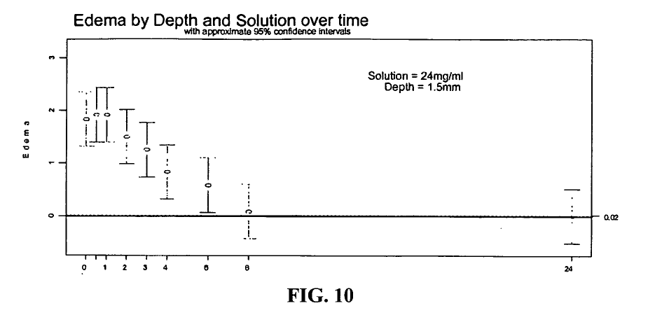

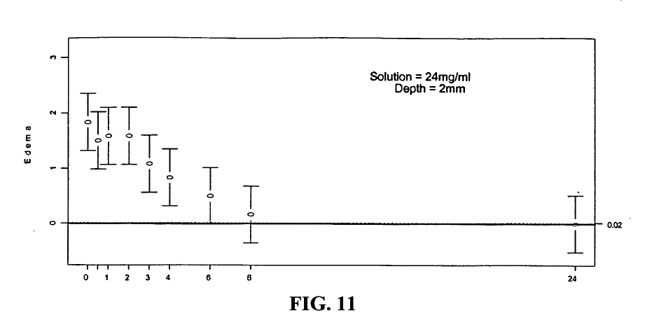

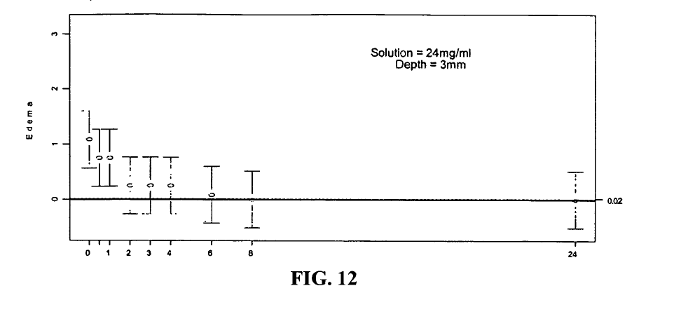

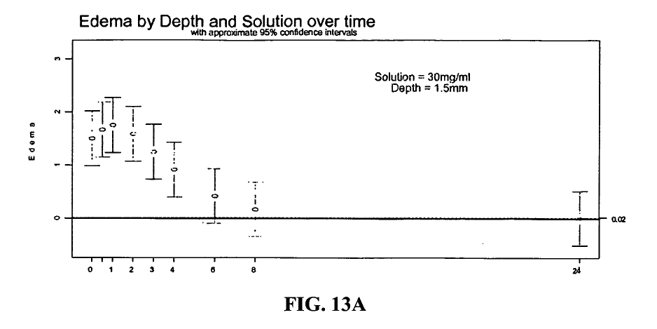

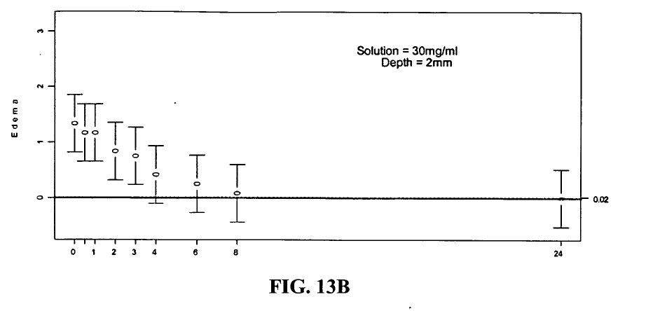

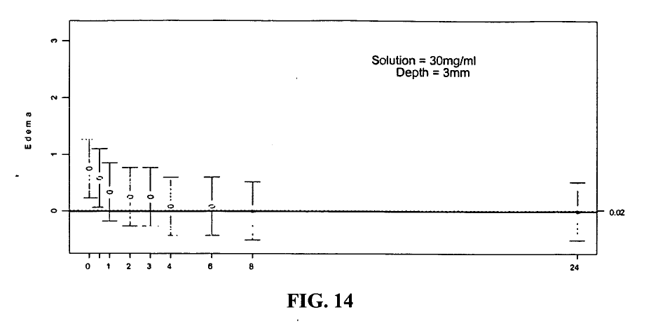

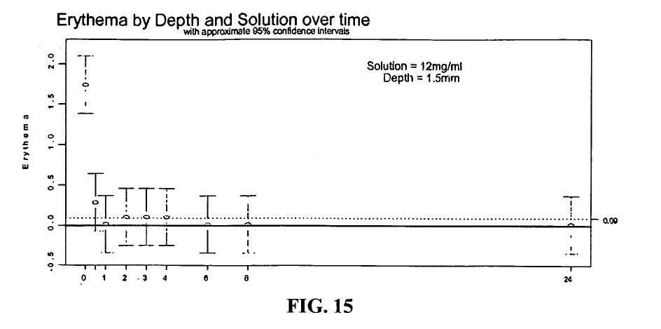

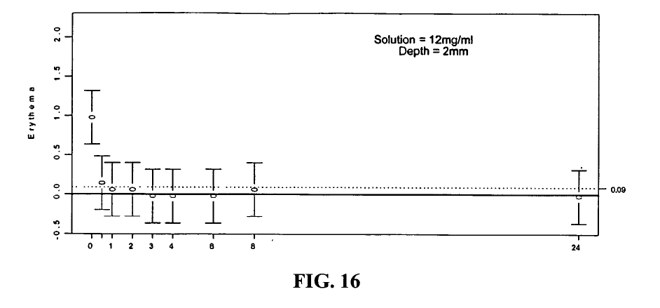

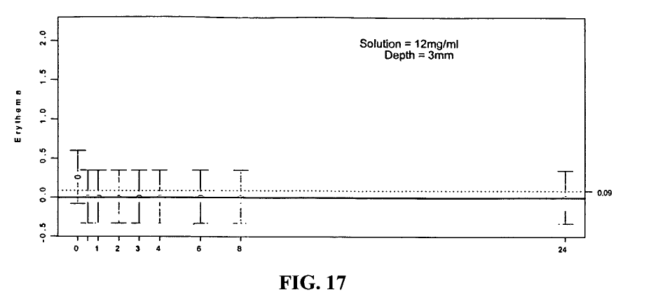

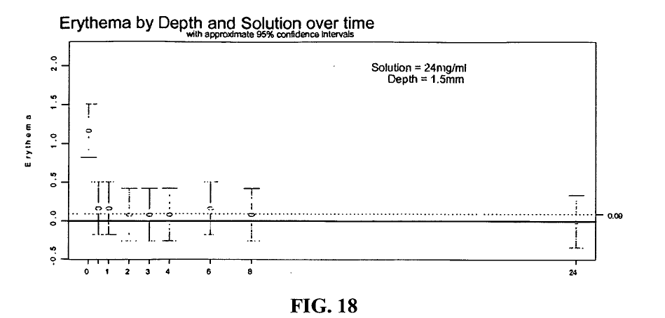

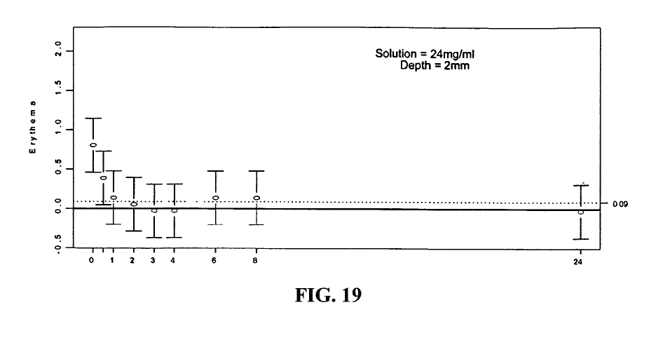

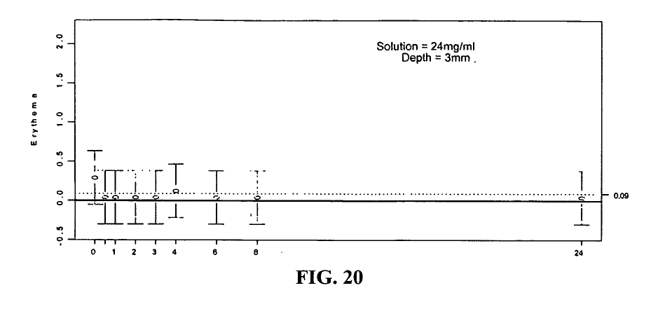

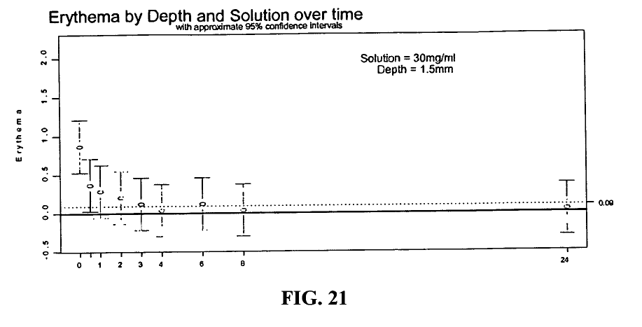

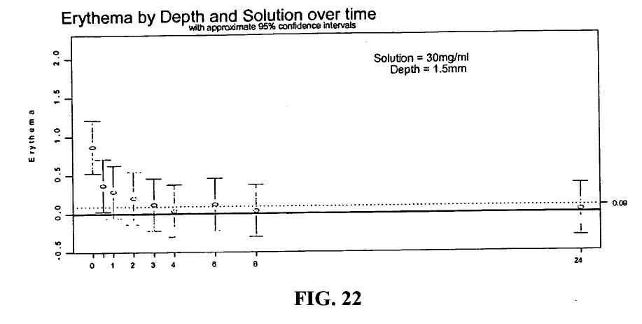

FIGS. 6-23 show the above significant main effects and interactions that involve the three factors under investigation. For bias calculations, an ANOVA was used followed by multiple comparisons (with approximate 95% confidence). Tables 3-6 summarize the biases between levels, over time, for the significant factors and factor by time interactions. The significant differences are highlighted in yellow.

Table 3 presents the average edema differences between solutions at each given depth, for each time point. Table 4 presents the average edema differences between depths for each given solution, for each time point. The results are averaged over gauge since gauge was not a significant factor.

TABLE 3
Average Edema biases between Solutions at given Depths

| | | Solution | Time | | | | | | | | |
|---|---|---|---|---|---|---|---|---|---|---|---|
| Depth | | Bias | 0 | 0.5 | 1 | 2 | 3 | 4 | 6 | 8 | 24 |
| 1.5 | mm | 12-24 | 0.7 | 0.3 | 0.0 | 0.0 | −0.1 | −0.2 | −0.3 | 0.1 | 0.0 |
| | | | (0.0, 1.3) | (−0.3, 0.9) | (−0.6, 0.7) | (−0.6, 0.6) | (−0.8, 0.5) | (−0.9, 0.4) | (−0.9, 0.4) | (−0.5, 0.8) | (−0.7, 0.6) |
| | | 12-30 | 1.0 | 0.6 | 0.2 | −0.1 | −0.1 | −0.3 | −0.1 | 0.1 | 0.0 |
| | | | (0.4, 1.6) | (−0.1, 1.2) | (−0.4, 0.8) | (−0.7, 0.6) | (−0.8, 0.5) | (−1.0, 0.3) | (−0.7, 0.5) | (−0.6, 0−7) | (−0.7, 0.6) |
| | | 24-30 | 0.3 | 0.3 | 0.2 | −0.1 | 0.0 | −0.1 | 0.2 | −0.1 | 0.0 |
| | | | (−0.3, 1.0) | (−0.4, 0.9) | (−0.5, 0.8) | (−0.7, 0.5) | (−0.6, 0.6) | (−0.7, 0.5) | (−0.5, 0.8) | (−0.7, 0.5) | (−0.6, 0.6) |
| 2 | mm | 12-24 | 0.3 | 0.5 | 0.3 | 0.0 | −0.2 | −0.3 | −0.3 | −0.2 | 0.0 |
| | | | (−0.4, 0.9) | (−0.2, 1.2) | (−0.4, 0.9) | (−0.7, 0.7) | (−0.8, 0.5) | (−1.0, 0.3) | (−1.0, 0.3) | (−0.8, 0.5) | (−0.7, 0.7) |
| | | 12-30 | 0.8 | 0.8 | 0.7 | 0.8 | 0.2 | 0.1 | −0.1 | −0.1 | 0.0 |
| | | | (0.1, 1.4) | (0.2, 1.5) | (0.0, 1.4) | (0.1, 1.4) | (−0.5, 0.8) | (−0.6, 0.8) | (−0.8, 0.6) | (−0.8, 0.6) | (−0.7, 0.7) |
| | | 24-30 | 0.5 | 0.3 | 0.4 | 0.8 | 0.3 | 0.4 | 0.3 | 0.1 | 0.0 |
| | | | (−0.2, 1.2) | (−0.3, 1.0) | (−0.3, 1.1) | (0.1, 1.4) | (−0.3, 1.0) | (−0.3, 1.1) | (−0.4, 0.9) | (−0.6, 0.8) | (−0.7, 0.7) |
| 3 | mm | 12-24 | 0.5 | 0.3 | 0.0 | 0.2 | 0.0 | −0.1 | −0.1 | 0.0 | 0.0 |
| | | | (−0.1, 1.1) | (−0.2, 0.9) | (−0.6, 0.6) | (−0.4, 0.7) | (−0.6, 0.6) | (−0.6, 0.5) | (−0.6, 0.5) | (−0.6, 0.6) | (−0.6, 0.6) |
| | | 12-30 | 0.8 | 0.5 | 0.4 | 0.2 | 0.0 | 0.1 | −0.1 | 0.0 | 0.0 |
| | | | (0.3, 1.4) | (−0.1, 1.1) | (−0.1, 1.0) | (−0.4, 0.7) | (−0.6, 0.6) | (−0.5, 0.6) | (−0.6, 0.5) | (−0.6, 0.6) | (−0.6, 0.6) |
| | | 24-30 | 0.3 | 0.2 | 0.4 | 0.0 | 0.0 | 0.2 | 0.0 | 0.0 | 0.0 |
| | | | (−0.2, 0.9) | (−0.4, 0.7) | (−0.1, 1.0) | (−0.6, 0.6) | (−0.6, 0.6) | (−0.4, 0.7) | (−0.6, 0.6) | (−0.6, 0.6) | (−0.6, 0.6) |

TABLE 4
Average Edema biases between Depths for each Solution

| | Depth | Time | | | | | | | | |
|---|---|---|---|---|---|---|---|---|---|---|
| Solution | Bias | 0 | 0.5 | 1 | 2 | 3 | 4 | 6 | 8 | 24 |
| 12 mg/ml | 1.5-2 | 0.5 | 0.3 | 0.2 | 0.0 | 0.3 | 0.1 | 0.2 | 0.3 | 0.0 |
| | | (−0.3, 1.2) | (−0.5, 1.0) | (−0.6, 0.9) | (−0.8, 0.7) | (−0.5, 1.0) | (−0.6, 0.8) | (−0.5, 0.9) | (−0.5, 1.0) | (−0.7, 0.7) |
| | 1.5-3 | 1.0 | 1.2 | 1.2 | 1.1 | 0.9 | 0.5 | 0.4 | 0.3 | 0.0 |
| | | (0.2, 1.7) | (0.5, 1.9) | (0.5, 2.0) | (0.4, 1.8) | (0.2, 1.6) | (−0.3, 1.2) | (−0.4, 1.1) | (−0.5, 1.0) | (−0.7, 0.7) |
| | 2-3 | 0.5 | 0.9 | 1.1 | 1.2 | 0.7 | 0.3 | 0.2 | 0.0 | 0.0 |
| | | (−0.2, 1.2) | (0.2, 1.6) | (0.4, 1.8) | (0.5, 1.9) | (0.0, 1.4) | (−0.4, 1.0) | (−0.5, 0.9) | (−0.7, 0.7) | (−0.7, 0.7) |
| 24 mg/ml | 1.5-2 | 0.0 | 0.4 | 0.3 | −0.1 | 0.2 | 0.0 | 0.1 | −0.1 | 0.0 |
| | | (−0.6, 0.6) | (−0.2, 1.1) | (−0.3, 1.0) | (−0.7, 0.6) | (−0.5, 0.8) | (−0.6, 0.6) | (−0.6, 0.7) | (−0.7, 0.6) | (−0.6, 0.6) |
| | 1.5-3 | 0.8 | 1.2 | 1.2 | 1.3 | 1.0 | 0.6 | 0.5 | 0.1 | 0.0 |
| | | (0.1, 1.4) | (0.5, 1.8) | (0.5, 1.8) | (0.6, 1.9) | (0.4, 1.6) | (−0.1, 1.2) | (−0.1, 1.1) | (−0.6, 0.7) | (−0.6, 0.6) |
| | 2-3 | 0.8 | 0.8 | 0.8 | 1.3 | 0.8 | 0.6 | 0.4 | 0.2 | 0.0 |
| | | (0.1, 1.4) | (0.1, 1.4) | (0.2, 1.5) | (0.7, 2.0) | (0.2, 1.5) | (−0.1, 1.2) | (−0.2, 1.1) | (−0.5, 0.8) | (−0.6, 0.6) |
| 30 mg/ml | 1.5-2 | 0.2 | 0.5 | 0.6 | 0.8 | 0.5 | 0.5 | 0.2 | 0.1 | 0.0 |
| | | (−0.5, 0.8) | (−0.1, 1.1) | (0.0, 1.2) | (0.1, 1.4) | (−0.1, 1.1) | (−0.1, 1.1) | (−0.5, 0.8) | (−0.5, 0.7) | (−0.6, 0.6) |
| | 1.5-3 | 0.8 | 1.1 | 1.4 | 1.3 | 1.0 | 0.8 | 0.3 | 0.2 | 0.0 |
| | | (0.1, 1.4) | (0.5, 1.7) | (0.8, 2.0) | (0.7, 2.0) | (0.4, 1.6) | (0.2, 1.5) | (−0.3, 1.0) | (−0.5, 0.8) | (−0.6, 0.6) |
| | 2-3 | 0.6 | 0.6 | 0.8 | 0.6 | 0.5 | 0.3 | 0.2 | 0.1 | 0.0 |
| | | (0.0, 1.2) | (0.0, 1.2) | (0.2, 1.5) | (0.0, 1.2) | (−0.1, 1.1) | (−0.3, 1.0) | (−0.5, 0.8) | (−0.5, 0.7) | (−0.6, 0.6) |

Tables 5, 6 and 7 present the average erythema differences at time 0 and 0.5, because there are no significant erythema biases past 0.5 hour.

TABLE 5
Average Erythema biases between Depths

Time

| Depth | 0 h | 0.5 h |
|---|---|---|
| 1.5 mm-2 mm | 0.4 | −0.1 |
| | (0.0, 0.7) | (−0.4, 0.2) |
| 1.5 mm-3 mm | 1.0 | 0.2 |
| | (0.6, 1.3) | (−0.1, 0.5) |
| 2 mm-3 mm | 0.6 | 0.3 |
| | (0.2, 0.9) | (0.0, 0.6) |

TABLE 6
Average Erythema biases between Solutions for given Gauges

Time

| Gauge | Solution Bias | 0 h | 0.5 h |
|---|---|---|---|
| 30 | 12 mg/ml-24 mg/ml | 0.3 | 0.1 |
| | | (−0.3, 0.9) | (−0.4, 0.6) |
| | 12 mg/ml-30 mg/ml | 0.7 | 0.0 |
| | | (0.1, 1.3) | (−0.4, 0.5) |
| | 24 mg/ml-30 mg/ml | 0.4 | 0.0 |
| | | (−0.2, 0.9) | (−0.5, 0.4) |
| 34 | 12 mg/ml-24 mg/ml | 0.1 | −0.2 |
| | | (−0.5, 0.7) | (−0.6, 0.3) |
| | 12 mg/ml-30 mg/ml | 0.1 | −0.3 |
| | | (−0.5, 0.7) | (−0.8, 0.2) |
| | 24 mg/ml-30 mg/ml | 0.0 | −0.1 |
| | | (−0.6, 0.6) | (−0.6, 0.3) |

TABLE 7
Average Erythema biases between Gauges for given Solutions

Time

| Depth | Gauge Bias | 0 h | 0.5 h |
|---|---|---|---|
| 12 mg/ml | 30-34 | −0.2 | 0.1 |
| | | (−0.7, 0.3) | (−0.3, 0.5) |
| 24 mg/ml | 30-34 | −0.5 | −0.2 |
| | | (−1.0, 0.1) | (−0.6, 0.2) |
| 30 mg/ml | 30-34 | −0.8 | −0.2 |
| | | (−1.4, −0.3) | (−0.7, 0.2) |

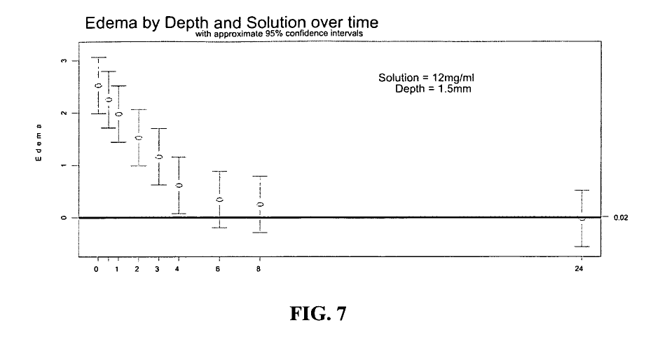

FIGS. 7-23 show the edema and erythema average scores (with approximate 95% CI) over time for each level of the significant factor by time interactions. When the confidence interval overlaps 0, the score is no longer significantly different from 0. Note that control conditions were run and results gave a mean edema score of 0.01 (95% upper bound of 0.02) and a mean erythema score of 0.06 (95% upper bound of 0.09). When an edema confidence interval below overlaps 0.02, and when an erythema confidence interval below overlap 0.09, the respective scores are no longer different from the control conditions.

TABLE 8
FINAL SUMMARY

edema ˜ (pig + Gauge + Depth + Solution + Time)\hat{ }2
>anova(edemanum.glm, test = “Chi”)
Analysis of Deviance Table
Poisson model
Response: edema
Terms added sequentially (first to last)

| | Df | Deviance Resid. | Df | Resid. Dev | Pr(Chi) |
|---|---|---|---|---|---|
| NULL | | | 962 | 1113.2634 | |
| pig | 11 | 36.11906 | 951 | 1077.1444 | 0.00016169 |
| Gauge | 1 | 2.04007 | 950 | 1075.1043 | 0.15320302 |
| Depth | 2 | 126.98221 | 948 | 948.1221 | 0.00000000 |
| Solution | 2 | 12.51437 | 946 | 935.6077 | 0.00191663 |
| Time | 1 | 488.26285 | 945 | 447.3449 | 0.00000000 |
| pig: Gauge | 11 | 10.22282 | 934 | 437.1221 | 0.51046258 |
| pig: Depth | 22 | 62.60690 | 912 | 374.5152 | 0.00000912 |
| pig: Solution | 22 | 17.76699 | 890 | 356.7482 | 0.71971055 |
| pig: Time | 11 | 17.67699 | 879 | 339.0712 | 0.08938470 |
| Gauge: Depth | 2 | 1.55888 | 877 | 337.5123 | 0.45866185 |
| Gauge: Solution | 2 | 0.42349 | 875 | 337.0888 | 0.80917121 |
| Gauge: Time | 1 | 0.86635 | 874 | 336.2225 | 0.35196659 |
| Depth: Solution | 4 | 4.31182 | 870 | 331.9106 | 0.36544695 |
| Depth: Time | 2 | 15.72785 | 868 | 316.1828 | 0.00038436 |
| Solution: Time | 2 | 5.06660 | 866 | 311.1162 | 0.07939677 |

step.edemanum$anova
Stepwise Model Path
Analysis of Deviance Table
Initial Model:
edema ˜ (pig + Gauge + Depth + Solution + Time)\hat{ }2
Final Model:
edema ˜ pig + Depth + Solution + Time + pig: Depth + Depth: Time + Solution:
Time
anova(edema.glm, test = “Chi”)
Analysis of Deviance Table
Poisson model
Response: edema
Terms added sequentially (first to last)

| | Df | Deviance Resid. | Df | Resid. Dev | Pr(Chi) |
|---|---|---|---|---|---|
| NULL | | | 962 | 1113.2634 | |
| pig | 11 | 36.11906 | 951 | 1077.1444 | 0.000161691 |
| Depth | 2 | 128.24849 | 949 | 948.8959 | 0.000000000 |
| Solution | 2 | 12.46509 | 947 | 936.4308 | 0.001964442 |
| Time | 1 | 488.26285 | 946 | 448.1679 | 0.000000000 |
| pig: Depth | 22 | 60.10535 | 924 | 388.0626 | 0.000021561 |
| Depth: Time | 2 | 15.17615 | 922 | 372.8864 | 0.000506455 |
| Solution: Time | 2 | 6.56086 | 920 | 366.3256 | 0.037612038 |

anova(erythema.glm, test = “Chi”)
erythema ˜ (pig + Gauge + Depth + Solution + Time)\hat{ }2
Analysis of Deviance Table
Poisson model
Response: erythema
Terms added sequentially (first to last)

| | Df | Deviance Resid. | Df | Resid. Dev | Pr(Chi) |
|---|---|---|---|---|---|
| NULL | | | 962 | 641.01867 | |
| pig | 11 | 55.43734 | 951 | 585.58133 | 0.00000006 |
| Gauge | 1 | 15.21827 | 950 | 570.36306 | 0.00009577 |
| Depth | 2 | 37.46783 | 948 | 532.89523 | 0.00000001 |
| Solution | 2 | 0.37531 | 946 | 532.51991 | 0.82889868 |
| Time | 1 | 143.98682 | 945 | 388.53309 | 0.00000000 |
| pig: Gauge | 11 | 18.52382 | 934 | 370.00927 | 0.07019527 |
| pig: Depth | 22 | 35.53102 | 912 | 334.47825 | 0.03406865 |
| pig: Solution | 22 | 40.24445 | 890 | 294.23379 | 0.01012215 |
| pig: Time | 11 | 52.83727 | 879 | 241.39652 | 0.00000019 |
| Gauge: Depth | 2 | 6.93774 | 877 | 234.45878 | 0.03115216 |
| Gauge: Solution | 2 | 8.59976 | 875 | 225.85902 | 0.01357022 |
| Gauge: Time | 1 | 4.15630 | 874 | 221.70272 | 0.04147987 |
| Depth: Solution | 4 | 17.11720 | 870 | 204.58552 | 0.00183417 |
| Depth: Time | 2 | 2.49649 | 868 | 202.08903 | 0.28700864 |
| Solution: Time | 2 | 8.07557 | 866 | 194.01346 | 0.01763650 |

Stepwise Model Path
Analysis of Deviance Table
Initial Model:
erythema ˜ (pig + Gauge + Depth + Solution + Time)\hat{ }2
Final Model:
erythema ˜ pig + Gauge + Depth + Solution + Time + pig: Depth +
pig: Solution + pig: Time + Gauge: Solution + Solution: Time
>anova(erythema.glm, test = “Chi”)
Analysis of Deviance Table
Poisson model
Response: erythema
Terms added sequentially (first to last)

| | Df | Deviance Resid. | Df | Resid. Dev | Pr(Chi) |
|---|---|---|---|---|---|
| NULL | | | 962 | 641.01867 | |
| pig | 11 | 55.43734 | 951 | 585.58133 | 0.00000006 |
| Gauge | 1 | 15.21827 | 950 | 570.36306 | 0.00009577 |
| Depth | 2 | 37.46783 | 948 | 532.89523 | 0.00000001 |
| Solution | 2 | 0.37531 | 946 | 532.51991 | 0.82889868 |
| Time | 1 | 143.98682 | 945 | 388.53309 | 0.00000000 |
| pig: Depth | 22 | 34.14536 | 923 | 354.38773 | 0.04747748 |
| pig: Solution | 22 | 39.85769 | 901 | 314.53003 | 0.01123300 |
| pig: Time | 11 | 52.82595 | 890 | 261.70408 | 0.00000019 |
| Gauge: Solution | 2 | 8.17012 | 888 | 253.53396 | 0.01682213 |
| Solution: Time | 2 | 9.18626 | 886 | 244.34771 | 0.01012115 |

6.2 Experimental Study for PK Determination

A crossover PK study was performed in Yucatan miniswine to compare the systemic availability of sumatriptan upon ID and junctional administration of marketed vs. one of the new formulations (30 mg/mL) as well as to determine any effects of device (including needle depth) and injection technique. Another objective of this study was to compare delivery formulations of sumatriptan succinate via the delivery methods of the invention to conventional delivery methods. Sumatriptan succinate is conventionally delivered to the SC compartment of skin. Conventional delivery to the SC compartment requires delivery at a depth of at least 5 mm, typically ranging from 8 mm to 13 mm.

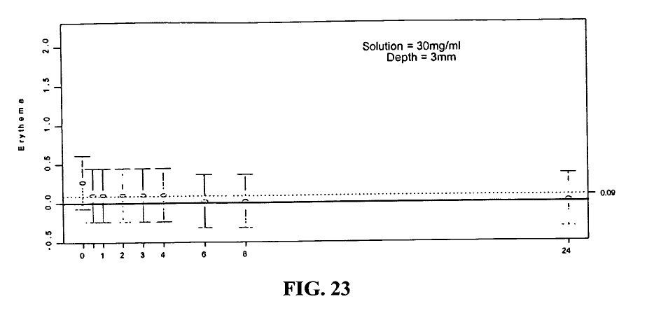

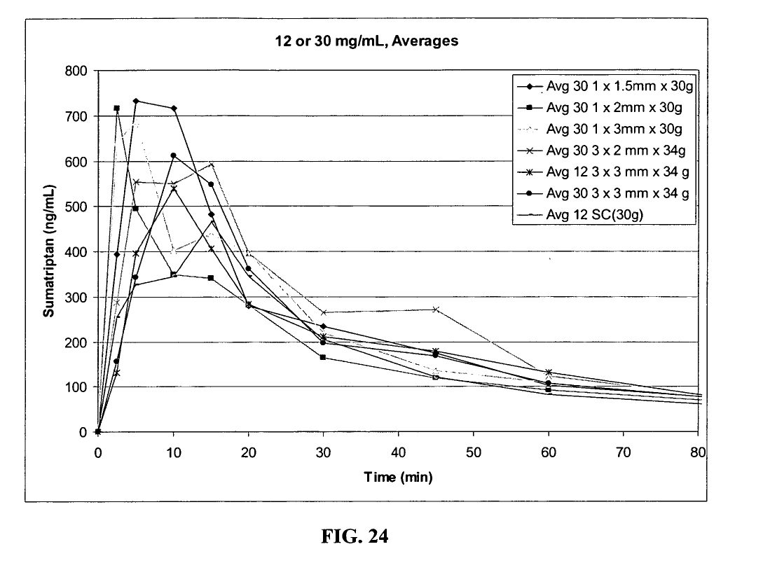

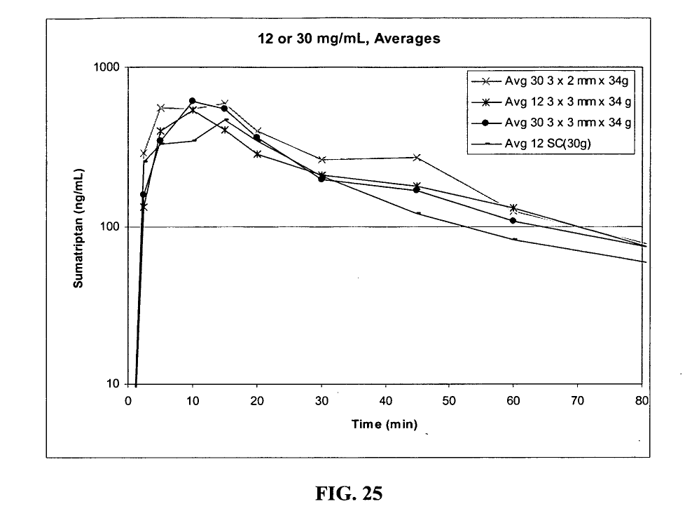

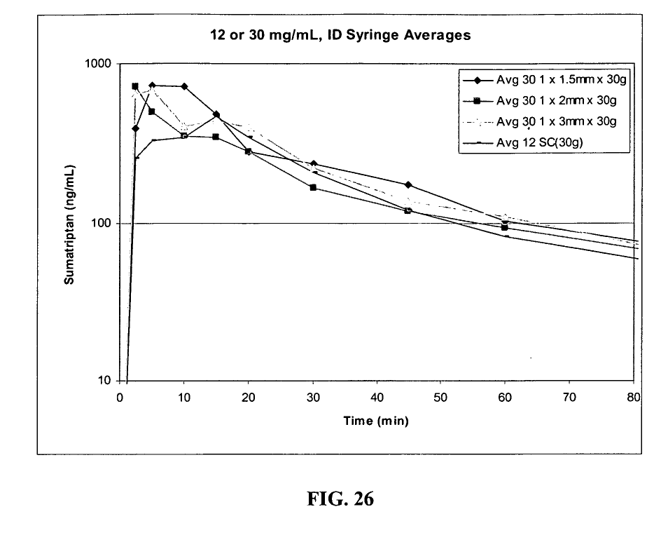

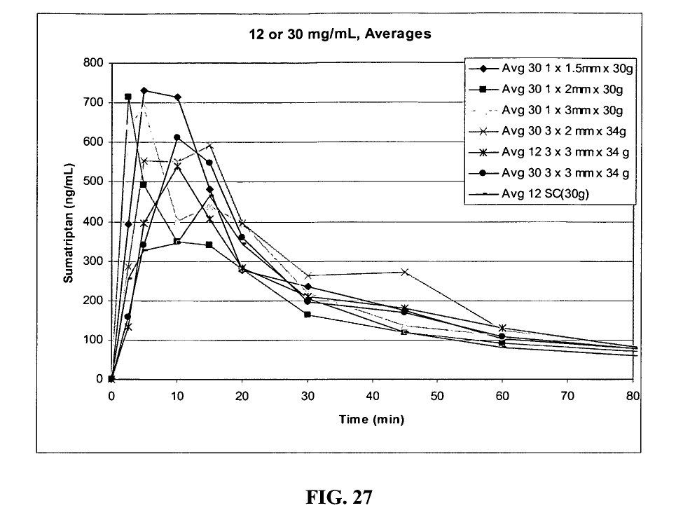

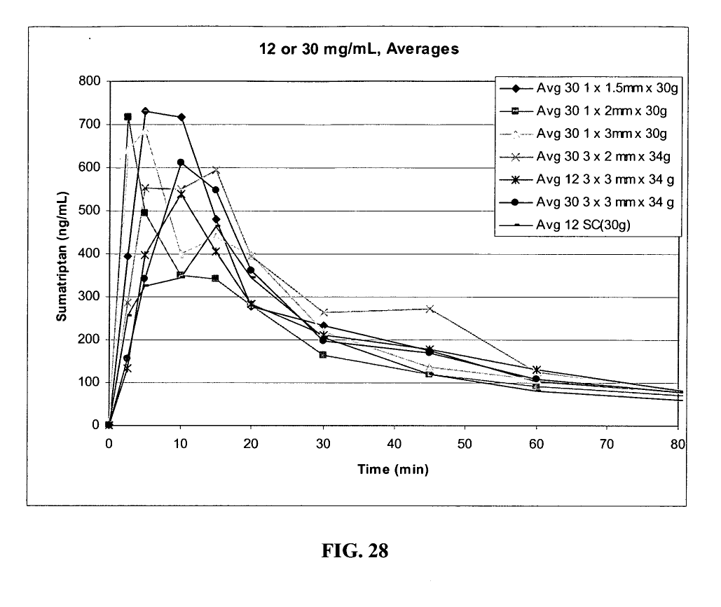

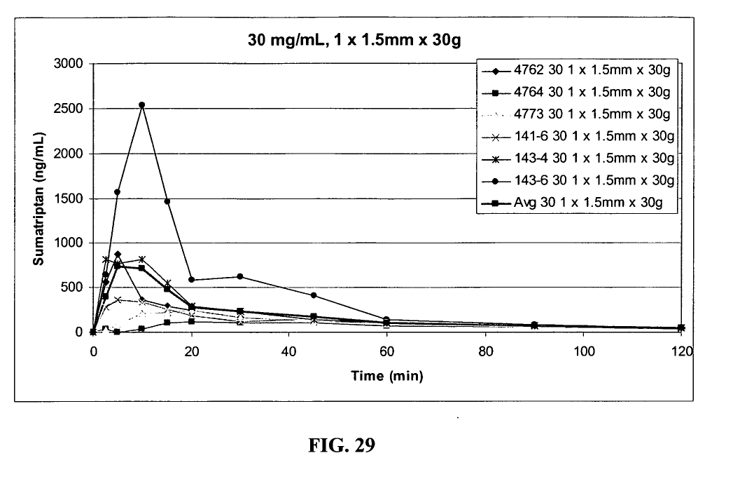

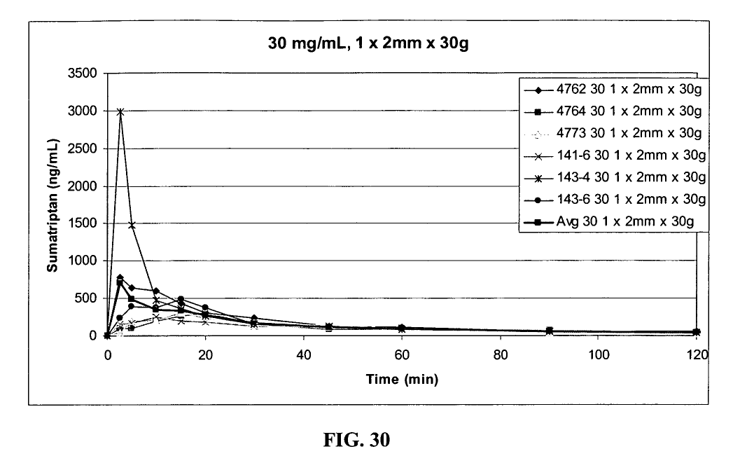

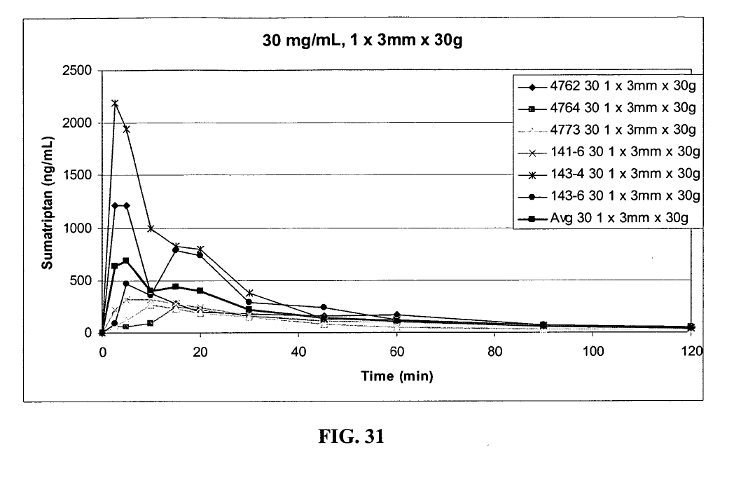

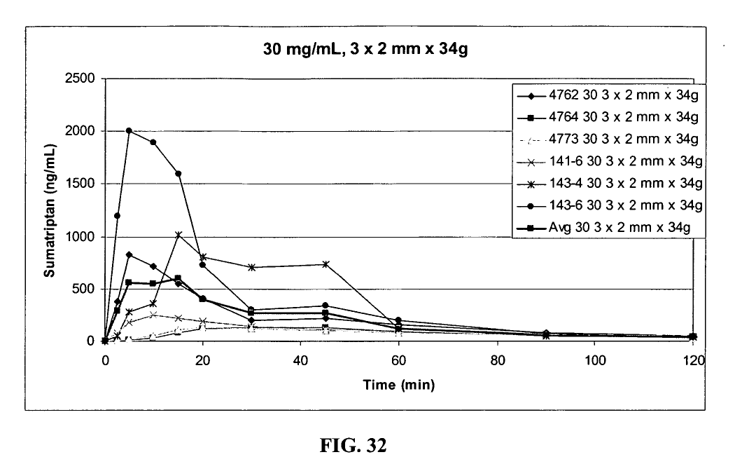

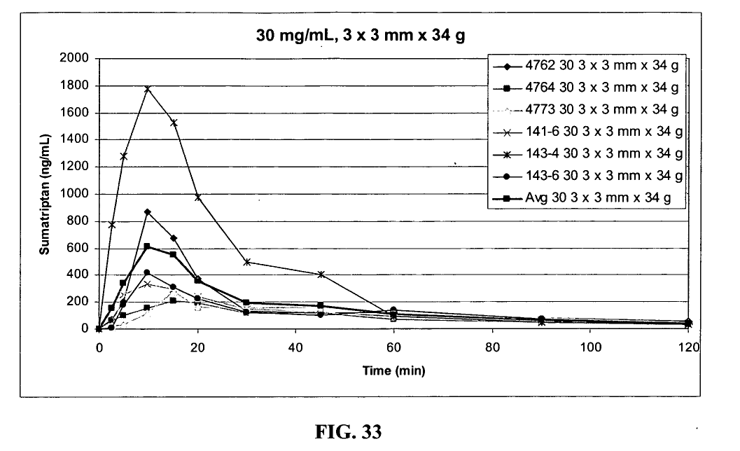

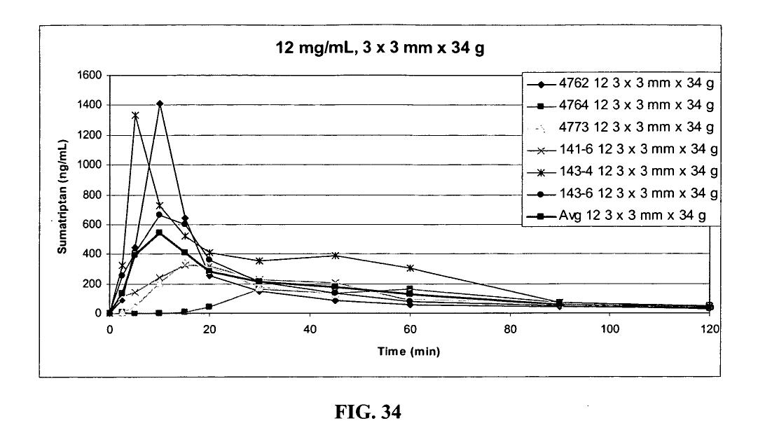

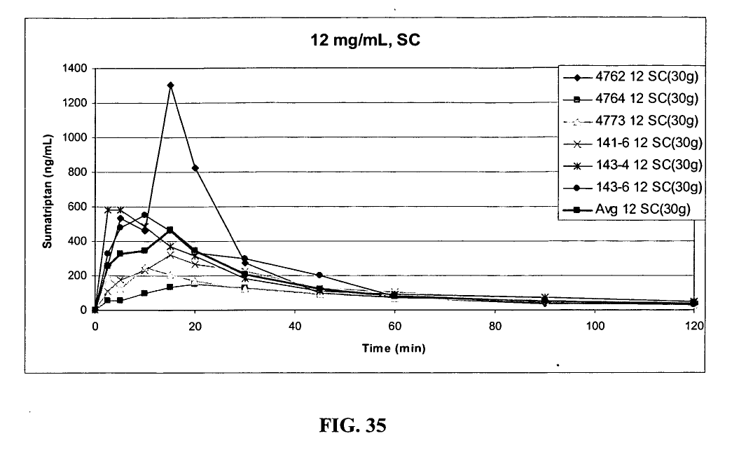

Timed blood samples were analyzed for sumatriptan content using an LC/MS/MS assay, which was previously validated for use with swine plasma. Average plasma levels profiles for the various conditions are shown in FIGS. 24 and 25. In the case of syringe based injection, peak plasma levels of sumatriptan are achieved within 5 minutes using the high concentration formulation paired with any needle length. Conversely the SC injection does not achieve maximal plasma levels until 15 minutes. This data would indicate a more rapid onset of action for the administered drug solution. Maximum concentration levels at early time points also appear elevated for Intradermal and/or junctional administration. A summary of the results is shown in FIGS. 23-35.

In the case of catheter based Intradermal and/or junctional sumatriptan infusions, peak levels were achieved within 10 minutes for the two infusions at 3 mm depth and at 15 minutes for the 2 mm infusion. In this instance, Intradermal and/or junctional infusion more closely resembles current SC injection. However, it should be noted that complete instillation of the total drug dose was initiated at time 0, but not completed until t=2 minutes. This still implies a faster onset of action via the Intradermal and/or junctional route.

6.3 Pharmacokinetic Analysis

A PK analysis was performed by observing the Tₘₐₓ and Cₘₐₓ and calculating AUC (Area Under the Curve) total, AUC 10 minutes, relative F total (bioavailability) compared to SC, relative F 10 minutes compared to SC for each animal in the study and averaged for each condition.

AUC was calculated using the trapezoidal method:

$$AUC = \frac{{C}_{n} + {C}_{n + 1}}{2} * {T}_{n + 1} - {T}_{n}$$

The AUC 10 minutes are the AUC values summed up to the 10 minute time point of the condition and the AUC total is the AUC values summed for the entire condition for the animal.

Percent (%) relative Bioavailability (F) for each dosing condition was calculated compared to the SC condition:

$$\% rel . {F}_{cond} = \frac{{AUC}_{cond}}{{AUC}_{SC}} * 100$$

The % relative F total for each condition used the AUC total, and the % relative F 10 minutes used the AUC 10 minutes in calculating values. Calculations are summarized in Table 9 below. Additional References used for the calculations above are: “Final Report, Study # 109335, The Determination of Sumatriptan in Pig Plasma, Biovail Contract Research, Toronto, Ontario” and “Applied Biopharmaceutics and Pharmacokinetics, fourth edition. Leon Shargel, Andrew Yu. McGraw-Hill, New York. 1999”.

TABLE 9
Summary of Calculations

| | | | | AUC 10 | | |
|---|---|---|---|---|---|---|
| | T max | C max | AUC total | min. (ng | rel. F | rel. F |
| | (minutes) | (ng/ml) | (ng min/ml) | min/ml) | Total (%) | 10 min (%) |
| Condition | Std Dev | Std Dev | Std Dev | Std Dev | Std Dev | Std Dev |
| IV, | 2.500 | 660.833 | 16626.000 | 3585.500 | 70.719 | 131.820 |
| 12 mg/ml | 0.000 | 157.164 | 809.979 | 650.061 | 3.445 | 23.899 |
| SC, | 12.500 | 526.667 | 23510.000 | 2720.000 | 100.000 | 100.000 |
| 12 mg/ml | 5.244 | 416.024 | 5731.290 | 1713.342 | 24.378 | 62.991 |
| 1 × 1.5 mm × 30 g, | 10.417 | 824.000 | 27696.167 | 5080.333 | 117.806 | 186.777 |
| 0 mg/ml | 7.813 | 891.994 | 15243.746 | 5006.070 | 64.839 | 184.047 |
| 1 × 2 mm × 30 g, | 10.833 | 849.333 | 23548.167 | 4515.000 | 100.162 | 165.993 |
| 30 mg/ml | 7.188 | 1061.609 | 5626.949 | 5041.551 | 23.934 | 185.351 |
| 1 × 3 mm × 30 g, | 9.167 | 836.667 | 27335.167 | 5181.333 | 116.270 | 190.490 |
| 30 mg/ml | 5.627 | 765.193 | 11245.867 | 5666.261 | 47.834 | 208.318 |
| 3 × 2 mm × 34 g, | 14.167 | 725.667 | 30006.000 | 4169.000 | 127.631 | 153.272 |
| 30 mg/ml | 9.704 | 730.060 | 16735.920 | 5813.594 | 71.186 | 213.735 |
| 3 × 3 mm × 34 g, | 11.667 | 648.667 | 25947.667 | 3204.333 | 110.369 | 117.806 |
| 30 mg/ml | 2.582 | 599.197 | 13295.235 | 4005.895 | 56.551 | 147.276 |
| 3 × 3 mm × 34 g, | 19.167 | 705.833 | 25642.833 | 3167.000 | 109.072 | 116.434 |
| 12 mg/ml | 20.351 | 541.995 | 8893.721 | 2986.948 | 37.830 | 109.814 |

The comparison of condition 3×3 mm×34 g 30 mg/ml to 3×3 mm×34 g 12 mg/ml demonstrates equivalence between the 30 mg/ml formulation and the commercially available 12 mg/ml formulation of sumatriptan. The only difference between the two conditions was the Tₘₐₓ because the 30 mg/ml formulation was delivered in a 2 minute metered bolus and the 12 mg/ml was a 5 minutes metered bolus. This means that increasing the high concentrations of sumatriptan do not have a deleterious effect on the relative bioavailability and absorption mechanisms. This also indicates that intradermal and/or junctional sumatriptan administration of various dose volumes and concentrations allows rapid uptake and distribution. The results indicate that the 30 mg/ml sumatriptan formulation is acceptable for use.

The Intradermal and/or junctional delivery of sumatriptan via rapid bolus and metered bolus has better than or equal to relative bioavailability than a SC injection of sumatriptan. This was demonstrated in the rel. F total and rel. F 10 minutes. The better rel. F 10 minutes indicates a faster systemic availability of sumatriptan after administration, which potentially equates to a faster onset of therapy and relief from symptoms, and thus, the methods of the invention are expected to provide a better therapeutic outcome.

The methods and compositions described above are simply representative of aspects of the invention. All of the patents, patent applications and publications referred to in this application are incorporated herein in their entireties. However, citation or identification of any reference in this application is not an admission that such reference is available as prior art to this invention. The full scope of the invention is better understood with reference to the appended claims.

1. A formulation for parenteral administration comprising a triptan compound, or a pharmaceutically acceptable salt thereof, and a pharmaceutically acceptable carrier or excipient, wherein the formulation contains no NaCl.

2. The formulation of claim 1, wherein the triptan compound is sumatriptan or sumatriptan succinate.

3. The formulation of claim 1, wherein the triptan compound is almotriptan malate, rizatriptan benzoate, zolmitriptan, or naratriptan hydrochloride.

4. The formulation of claim 1, wherein the excipient is sugar- or carbohydrate-based tonicity agent.

5. The formulation of claim 4, wherein the tonicity agent is mannitol.

6. The formulation of claim 4, wherein the tonicity agent is dextrose.

7. The formulation of claim 4, wherein the tonicity agent is sorbitol.

8. The formulation of claim 2, wherein the sumatriptan succinate is present at a concentration of from about 20 mg/ml to about 40 mg/ml.

9. The formulation of claim 8, wherein the sumatriptan succinate is present at a concentration of from about 20 mg/ml to about 30 mg/ml.

10. The formulation of claim 2, wherein the sumatriptan succinate is present at a concentration of about 24 mg/ml.

11. The formulation of claim 10, which comprises about 33.6 mg sumatriptan succinate, about 0.71 mg dibasic sodium phosphate anhydrous, and about 19.49 mg mannitol, and wherein the pH of the formulation is adjusted to about 5.5.

12. The formulation of claim 2, wherein the sumatriptan succinate is present at a concentration of about 30 mg/ml.

13. The formulation of claim 12, which comprises about 42.0 mg sumatriptan succinate, about 0.71 mg dibasic sodium phosphate anhydrous, and about 12.21 mg mannitol, and wherein the pH of the formulation is adjusted to about 5.5.

14. A method of treating, preventing, or managing pain comprising administering by injection into the skin of a patient in need of such treatment, prevention, or management a formulation comprising sumatriptan, or a pharmaceutically acceptable salt thereof, and a pharmaceutically acceptable carrier or excipient, wherein the formulation contains no NaCl.

15. The method of claim 14, wherein the formulation is injected into the intradermal and/or junctional compartment of the skin.

16. The method of claim 14, wherein the pharmaceutically acceptable salt is sumatriptan succinate.

17. The method of claim 14, wherein the excipient is sugar- or carbohydrate-based tonicity agent.

18. The method of claim 17, wherein the tonicity agent is mannitol.

19. The method of claim 17, wherein the tonicity agent is dextrose.

20. The method of claim 17, wherein the tonicity agent is sorbitol.

21. The method of claim 16, wherein the sumatriptan succinate is present at a concentration of from about 20 mg/ml to about 40 mg/ml.

22. The method of claim 16, wherein the sumatriptan succinate is present at a concentration of from about 20 mg/ml to about 30 mg/ml.

23. The method of claim 16, wherein the sumatriptan succinate is present at a concentration of about 24 mg/ml.

24. The method of claim 23, which comprises about 33.6 mg sumatriptan succinate, about 0.71 mg dibasic sodium phosphate anhydrous, and about 19.49 mg mannitol, and wherein the pH of the formulation is adjusted to about 5.5.

25. The method of claim 16, wherein the sumatriptan succinate is present at a concentration of about 30 mg/ml.

26. The method of claim 25, which comprises about 42.0 mg sumatriptan succinate, about 0.71 mg dibasic sodium phosphate anhydrous, and about 12.21 mg mannitol, and wherein the pH of the formulation is adjusted to about 5.5.

27. The method of claim 14, wherein the pain is nociceptive pain, neuropathic pain, acute pain, chronic pain, osteoarthritis, rheumatoid arthritis or tendonitis, myofascial pain, visceral pain, headache pain, reflex neurovascular dystrophy, reflex dystrophy, sympathetically maintained pain syndrome, causalgia, Sudeck atrophy of bone, algoneurodystrophy, shoulder hand syndrome, post-traumatic dystrophy, autonomic dysfunction, cancer-related pain, phantom limb pain, fibromyalgia, chronic fatigue syndrome, post-operative pain, spinal cord injury pain, central post-stroke pain, radiculopathy, allodynia, pain from hyperthermic or hypothermic conditions, diabetic neuropathy, luetic neuropathy, postherpetic neuralgia, trigeminal neuralgia, or painful neuropathy induced iatrogenically by vincristine, velcade or thalidomide.

28. The method of claim 27, wherein the headache pain is migraine headache pain.

29. The method of claim 14, wherein the administration is injection using a syringe.

30. The method of claim 29, wherein the injection is done by penetrating skin to a depth of about 0.5 mm to about 3 mm.

31. The method of claim 30, wherein the injection is done by penetrating skin to a depth of about 1 mm to about 3 mm.

32. The method of claim 30, wherein the injection is done by penetrating skin to a depth of about 2 mm to about 3 mm.

33. The method of claim 30, wherein the injection is done by penetrating skin to a depth of about 1.5 mm.

34. The method of claim 30, wherein the injection is done by penetrating skin to a depth of about 2 mm.

35. The method of claim 30, wherein the injection is done by penetrating skin to a depth of about 3 mm.

36. The method of claim 14, wherein sumatriptan, or a pharmaceutically acceptable salt thereof, is administered in combination with a second anti-pain agent.

37. The method of claim 36, wherein the second anti-pain agent is an antidepressant, an anticonvulsant, an antihypertensive, an anxiolytic, a calcium channel blocker, a muscle relaxant, an analgesic, an anti-inflammatory agent, a cox-2 inhibitor, an α-adrenergic receptor antagonist, ketamine, an anesthetic, an immunomodulatory agent, an immunosuppressive agent, a corticosteroid, hyperbaric oxygen, or an NMDA antagonist.

38. The method of claim 36, wherein sumatriptan, or a pharmaceutically acceptable salt thereof, and the second anti-pain agent are simultaneously administered.

39. The method of claim 36, wherein sumatriptan, or a pharmaceutically acceptable salt thereof, and the second anti-pain agent are sequentially administered.
